(19) United States
(12) Patent Application Publication
(10) Pub. No.: US 2026/0034073 A1
(43) Pub. Date: Feb. 5, 2026

(54) COMBINATION THERAPY OF COENZYME Q10 AND RADIATION FOR TREATMENT OF GLIOMA

(71) Applicant: BPGbio, Inc., Framingham, MA (US)

(72) Inventors: Niven Rajin Narain, Cambridge, MA (US); Rangaprasad Sarangarajan, Boylston, MA (US)

(21) Appl. No.: 19/089,878

(22) Filed: Mar. 25, 2025

Related U.S. Application Data
(63) Continuation of application No. 17/100,674, filed on Nov. 20, 2020, now abandoned.
(60) Provisional application No. 62/938,077, filed on Nov. 20, 2019.

(51) Int. Cl. A61K 31/12; A61K 9/00; A61K 31/122; A61K 31/495; A61K 39/395; A61N 1/40; A61N 5/10; A61P 35/00

(52) U.S. Cl. CPC A61K 31/12; A61K 9/0019; A61K 31/122; A61K 31/495; A61K 39/3955; A61N 1/40; A61N 5/10; A61P 35/00; A61N 2005/1098

(57) ABSTRACT

The invention provides methods and compositions for treatment of a subject with a glioma. The methods comprise administering a composition comprising Coenzyme Q10 to the subject by continuous intravenous infusion; and administering radiation therapy to the subject. The composition comprising Coenzyme Q10 may be administered to the subject by continuous intravenous infusion for at least 24 hours before the radiation therapy is initiated.

### RELATED APPLICATIONS

This application is a Continuation of U.S. Non-Provisional patent application Ser. No. 17/100,674 filed on Nov. 20, 2020 which, in turn, claims priority to U.S. Provisional Patent Application No. 62/938,077 filed on Nov. 20, 2019, the contents of which are incorporated herein in their entirety.

### BACKGROUND

High Grade Gliomas, including anaplastic astrocytomas, anaplastic oligodendrogliomas and glioblastomas (GBM), are the most common and most aggressive primary brain tumors. Prognosis for patients with high-grade gliomas remains poor. The estimated median survival for patients with GBM is between 12 to 18 months. Recurrence after initial therapy with temozolomide and radiation is nearly universal. Since May 2009, the majority of patients in the US with an initial recurrence of high-grade glioma receive bevacizumab, a monoclonal antibody against vascular endothelial growth factor (VEGF), which is thought to prevent angiogenesis in these highly vascular tumors. Bevacizumab has response rates from 32-62% and has improved overall median survival in patients with recurrent high-grade gliomas (Chamberlain M. C., 2009, Neurology 72(8): 772-3). However, the response is short lived, and nearly 100% of patients eventually progress despite bevacizumab. No chemotherapeutic agent administered following progression through bevacizumab has made a significant impact on survival (Shen et al., 2012, Journal of Cancer Therapy 3: 491-503). Therefore, a need exists for improved treatment regimens for glioma.

### SUMMARY OF THE INVENTION

In certain aspects, the disclosure relates to a method of treating a glioma in a subject comprising: (a) administering a composition comprising a Coenzyme Q10 compound (e.g. CoQ10) to the subject by continuous intravenous infusion; and (b) administering radiation therapy to the subject, wherein the composition comprising a Coenzyme Q10 compound (e.g. CoQ10) is administered to the subject by continuous intravenous infusion for at least 24 hours before the radiation therapy is initiated, thereby treating the glioma in the subject.

In certain embodiments, the composition comprising a Coenzyme Q10 compound (e.g. CoQ10) is administered to the subject by continuous intravenous infusion for at least 48 hours, at least 72 hours, or at least 96 hours before the radiation therapy is initiated. In certain embodiments, at least two doses of the composition comprising a Coenzyme Q10 compound (e.g. CoQ10) are administered to the subject by continuous intravenous infusion before the radiation therapy is initiated. In certain embodiments, each of the at least two doses is administered once per week. In certain embodiments, the radiation therapy is initiated at least one week after administration of the composition comprising a Coenzyme Q10 compound (e.g. CoQ10) is initiated. In certain embodiments, the radiation therapy is initiated at least two weeks after administration of the composition comprising a Coenzyme Q10 compound (e.g. CoQ10) is initiated.

In certain embodiments, the glioma is an anaplastic astrocytoma or a glioblastoma. In certain embodiments, the glioblastoma is a gliosarcoma. In certain embodiments, the subject has undergone surgery for the glioma before administration of the composition comprising a Coenzyme Q10 compound (e.g. CoQ10) is initiated. In certain embodiments, the subject has not been treated with an anticancer agent for the glioma before administration of the composition comprising a Coenzyme Q10 compound (e.g. CoQ10) is initiated. In certain embodiments, the anticancer agent is selected from temozolomide (TMZ) and bevacizumab. In certain embodiments, the subject has not been treated with radiation therapy for the glioma before administration of the composition comprising a Coenzyme Q10 compound (e.g. CoQ10) is initiated.

In certain embodiments, the subject demonstrates a clinical benefit as a result of administration of the composition comprising a Coenzyme Q10 compound (e.g. CoQ10) compound and the radiation therapy. In certain embodiments, the clinical benefit is selected from the group consisting of stable disease per RECIST 1.1 criteria, partial response per RECIST 1.1 criteria, and complete response per RECIST 1.1 criteria. In certain embodiments, the subject achieves or maintains stable disease by RECIST 1.1 criteria as a result of administration of the composition comprising a Coenzyme Q10 compound (e.g. CoQ10) and the radiation therapy. In certain embodiments, the subject achieves or maintains a partial response by RECIST 1.1 criteria as a result of administration of the composition comprising the Coenzyme Q10 compound and the radiation therapy. In certain embodiments, the subject achieves or maintains a complete response by RECIST 1.1 criteria as a result of administration of the composition comprising the Coenzyme Q10 compound and the radiation therapy.

In certain embodiments, the glioma comprises a Stage I tumor. In certain embodiments, the glioma comprises a Stage II tumor. In certain embodiments, the glioma comprises a Stage III tumor. In certain embodiments, the glioma comprises a Stage IV tumor. In certain embodiments, the glioma is a low grade glioma. In certain embodiments, the glioma is a high grade glioma. In certain embodiments, the glioma is metastatic.

In certain embodiments, the Coenzyme Q10 is administered at a dose selected from the group consisting of at least 15.5 mg/kg/day (24 hours), at least 16.7 mg/kg/day (24 hours), at least 19.0 mg/kg/day (24 hours), at least 20.5 mg/kg/day (24 hours), at least 22.0 mg/kg/day (24 hours), at least 25.0 mg/kg/day (24 hours), at least 27.3 mg/kg/day (24 hours), at least 29.3 mg/kg/day (24 hours), at least 33.4 mg/kg/day (24 hours), at least 36.7 mg/kg/day (24 hours), or at least 34.1 mg/kg/day (24 hours). In certain embodiments, the Coenzyme Q10 is administered at a dose of at least 50 mg/kg/week, at least 66 mg/kg/week, at last 88 mg/kg/week or at least 110 mg/kg/week. In certain embodiments, the Coenzyme Q10 is administered at a dose of about 50 mg/kg/week, about 66 mg/kg/week, about 88 mg/kg/week or about 110 mg/kg/week. In certain embodiments, the Coenzyme Q10 is administered at a dose selected from the group consisting of at least 50 mg/kg/dose, at least 66 mg/kg/dose, at least 88 mg/kg/dose, or at least 110 mg/kg/dose. In certain embodiments, the Coenzyme Q10 is administered at a dose of about 50 mg/kg/dose, about 66 mg/kg/dose, about 88 mg/kg/dose or about 110 mg/kg/dose.

In certain embodiments, the Coenzyme Q10 is administered by continuous intravenous infusion for about 96 hours before the radiation therapy is initiated. In certain embodiments, at least 8 doses of the composition comprising a Coenzyme Q10 compound (e.g. CoQ10) are administered to the subject. In certain embodiments, the radiation therapy is administered at a total dose of 0.1 to 100 Gy. In certain embodiments, the radiation therapy is administered at a rate of 10 to 5000 cGy/min. In certain embodiments, the subject is human.

In certain embodiments, the method further comprises administering an additional cancer therapy to the subject. In certain embodiments, the additional cancer therapy comprises exposing the glioma to an alternating electrical field. In certain embodiments, the additional cancer therapy comprises administration of one or more anticancer agents to the subject. In certain embodiments, the one or more anticancer agents is selected from the group consisting of temozolomide (TMZ) and bevacizumab. In certain embodiments, the TMZ is administered a dose of about 75 mg/m² once per day. In certain embodiments, the TMZ is administered for at least 42 days. In certain embodiments, the method further comprises administering Vitamin K1 to the subject.

In certain embodiments, the subject exhibits an increase in one or more of overall survival and progression free survival relative to a subject that is administered the pharmaceutical composition comprising a Coenzyme Q10 compound (e.g. CoQ10) alone, or the radiation therapy alone. In certain embodiments, the composition comprising a Coenzyme Q10 compound (e.g. CoQ10) and the radiation therapy have a synergistic effect in treating the glioma.

In certain aspects the disclosure relates to a method of treating a glioma in a subject comprising administering to the subject: (a) a composition comprising a Coenzyme Q10 compound; and (b) radiation therapy, thereby treating the glioma in the subject. In some embodiments, the glioma is a glioblastoma. In some embodiments, the glioma is a refractory glioma. In some embodiments, the composition comprising the Coenzyme Q10 compound and the radiation therapy have a synergistic effect in treating the glioma. In some embodiments, the glioma is refractory to an anti-cancer agent selected from the group consisting of TMZ and bevacizumab. In some embodiments, the subject has failed treatment for the glioma with at least one additional anti-cancer therapy. In some embodiments, the at least one additional anti-cancer therapy is radiation therapy. In some embodiments, the at least one additional anti-cancer therapy is a chemotherapeutic agent. In some embodiments, the at least one additional anti-cancer therapy is an anti-angiogenic agent. In some embodiments, the at least one additional anti-cancer therapy is bevacizumab. In some embodiments, the failed treatment comprises tumor growth during or after treatment with the at least one additional anti-cancer agent.

In some embodiments, the subject demonstrates a clinical benefit as a result of administration of the composition comprising the Coenzyme Q10 compound and the radiation therapy. In some embodiments, the clinical benefit is selected from the group consisting of stable disease per RECIST 1.1 criteria, partial response per RECIST 1.1 criteria, and complete response per RECIST 1.1 criteria. In some embodiments, the subject achieves or maintains stable disease by RECIST 1.1 criteria as a result of administration of the composition comprising the Coenzyme Q10 compound and the radiation therapy. In some embodiments, the subject achieves or maintains a partial response by RECIST 1.1 criteria as a result of administration of the composition comprising the Coenzyme Q10 compound and the radiation therapy. In some embodiments, the subject achieves or maintains a complete response by RECIST 1.1 criteria as a result of administration of the composition comprising the Coenzyme Q10 compound and the radiation therapy.

In some embodiments, the glioma comprises a Stage I tumor. In some embodiments, the glioma comprises a Stage II tumor. In some embodiments, the glioma comprises a Stage III tumor. In some embodiments, the glioma comprises a Stage IV tumor. In some embodiments, the glioma is a low grade glioma. In some embodiments, the glioma is a high grade glioma. In some embodiments, the glioma is metastatic.

In some embodiments, the subject has further failed treatment with a chemotherapeutic agent selected from the group consisting of carmustine (BCNU), thalidomide, irinotecan, lomustine (CCNU), procarbazine, vincristine, and a platinum compound.

In some embodiments, the Coenzyme Q10 compound is Coenzyme Q10.

In some embodiments, the composition comprising the Coenzyme Q10 compound is administered one time per week, two times per week, or three times per week. In some embodiments, the Coenzyme Q10 is administered at a dose selected from the group consisting of at least 15.5 mg/kg/day (24 hours), at least 16.7 mg/kg/day (24 hours), at least 19.0 mg/kg/day (24 hours), at least 20.5 mg/kg/day (24 hours), at least 22.0 mg/kg/day (24 hours), at least 25.0 mg/kg/day (24 hours), at least 27.3 mg/kg/day (24 hours), at least 29.3 mg/kg/day (24 hours), at least 33.4 mg/kg/day (24 hours), at least 36.7 mg/kg/day (24 hours), at least 34.1 mg/kg/day (24 hours), at least 41.7 mg/kg/day (24 hours), at least 42.5 mg/kg/day (24 hours), at least 45.7 mg/kg/day (24 hours), at least 52.0 mg/kg/day (24 hours), at least 53.1 mg/kg/day (24 hours), at least 57 mg/kg/day (24 hours), at least 64.9 mg/kg/day (24 hours), at least 66.7 mg/kg/day (24 hours), at least 71.7 mg/kg/day (24 hours), at least 81.5 mg/kg/day (24 hours), at least 88.8 mg/kg/day (24 hours), at least 95.3 mg/kg/day (24 hours), at least 108.5 mg/kg/day (24 hours), at least 117.9 mg/kg/day (24 hours), at least 126.7 mg/kg/day (24 hours), at least 144.2 mg/kg/day (24 hours), at least 156.7 mg/kg/day (24 hours), at least 168.3 mg/kg/day (24 hours), at least 191.6 mg/kg/day (24 hours), at least 208.5 mg/kg/day (24 hours), at least 224 mg/kg/day (24 hours), and at least 254.9 mg/kg/day (24 hours).

In some embodiments, the Coenzyme Q10 is administered at a dose selected from the group consisting of at least 100 mg/kg/week, at least 132 mg/kg/week, at least 171 mg/kg/week, at least 215 mg/kg/week, at least 274 mg/kg/week, at least 430 mg/kg/week, at least 572 mg/kg/week, at least 760 mg/kg/week, at least 1010 mg/kg/week, and at least 1344 mg/kg/week. In some embodiments, the Coenzyme Q10 is administered at a dose selected from the group consisting of about 100 mg/kg/week, about 132 mg/kg/week, about 171 mg/kg/week, about 215 mg/kg/week, about 274 mg/kg/week, about 430 mg/kg/week, about 572 mg/kg/week, about 760 mg/kg/week, about 1010 mg/kg/week, and about 1344 mg/kg/week. In some embodiments, the Coenzyme Q10 is administered at a dose selected from the group consisting of at least 50 mg/kg/dose, at least 66 mg/kg/dose, at least 88 mg/kg/dose, at least 110 mg/kg/dose, at least 137 mg/kg/dose, at least 171 mg/kg/dose, at least 215 mg/kg/dose, at least 286 mg/kg/dose, at least 380 mg/kg/dose, at least 505 mg/kg/dose, and at least 672 mg/kg/dose. In some embodiments, the Coenzyme Q10 is administered at a dose selected from the group consisting of about 50 mg/kg/dose, about 66 mg/kg/dose, about 88 mg/kg/dose, about 110 mg/kg/dose, about mg/kg/dose, about 171 mg/kg/dose, about 215 mg/kg/dose, about 286 mg/kg/dose, about 380 mg/kg/dose, about 505 mg/kg/dose, and about 672 mg/kg/dose.

In some embodiments, the composition comprising the Coenzyme Q10 compound is administered by injection or infusion. In some embodiments, the composition comprising the Coenzyme Q10 compound is administered intravenously. In some embodiments, the composition comprising the Coenzyme Q10 is administered by continuous infusion. In some embodiments, the composition comprising the Coenzyme Q10 is administered by continuous infusion for at least 48 hours, at least 72 hours, at least 96 hours, at least 120 hours, or at least 144 hours. In some embodiments, the dose of Coenzyme Q10 is administered by continuous infusion over about 72 hours. In some embodiments, at least 12 doses, at least 15 doses, at least 26 doses, or at least 33 doses of the composition comprising the Coenzyme Q10 compound are administered to the subject. In some embodiments, the subject has failed 8 or fewer chemotherapeutic regimens. In some embodiments, the subject has failed 5 or fewer chemotherapeutic regimens. In some embodiments, the composition comprising the Coenzyme Q10 compound is administered orally.

In some embodiments, the radiation therapy is administered at a dose of 0.1 to 20 Gy. In some embodiments, the radiation therapy is administered at a rate of 10 to 5000 cGy/min. In some embodiments, the subject is human. In some embodiments, the composition comprising the Coenzyme Q10 compound is administered to the subject with an additional anti-cancer agent. In some embodiments, the additional anti-cancer agent is a chemotherapeutic agent. In some embodiments, the additional anti-cancer agent is an anti-angiogenic agent. In some embodiments, the additional anti-cancer agent is bevacizumab, TMZ or a combination thereof.

### BRIEF DESCRIPTION OF THE DRAWINGS

FIGS. 1A and 1B show that Coenzyme Q10 demonstrates an anti-tumor effect in an orthotopic glioma rat model. 10⁶ C6 glioma cells were implanted into the right striatum of Wistar rats. Four days after implantation, animals started to receive saline (n=32) or 50 mg/kg of Coenzyme Q10 (n=21), twice a day, i.p. for 45 days. Surviving animals remained in the study until Day 102 (A) Kaplan-Meier survival curve of rats during 45 days of treatment. Over 25% of rats treated with Coenzyme Q10 survived 45 days, considered long-term survival in this model (p<0.01, log rank test). (B) Serial MRI images of a long-term survivor (D27, D34, D102). The drug effect persisted after treatment was withdrawn, demonstrated by the continued shrinkage of the tumor over time (marked as dashed line). Far right panel is a coronal section of the long-term survivor.

FIGS. 2A-2F show differential induction of mitochondrial superoxide by Coenzyme Q10 in non-cancer vs. neoplastic cells. (A) Strategy of rodent co-culture experiments using glioma and non-tumor cells. The rodent cell co-culture consisted of fibroblastic NIH3T3 and glioma C6-GFP cells. (B) NIH3T3 and C6 cells were initially seeded in 1:1 ratio. After 72 h or 144 h incubation with Coenzyme Q10 (0 μM, 72 μM, 115 μM, 230 μM, 345 μM or 460μM), the number of C6 or NIH3T3 cells in co-culture is generated by flow-cytometry. (C) Images were taken at 72 hrs post-dosing (0 μM, 230 μM or 460 μM) using a Leica CTR5000. (D-G) NIH3T3 and C6 cells co-cultured with certain conc. of Coenzyme Q10 for 144 hours were subjected to imaging and flow-cytometry. Superoxide level is indicated by Mitosox staining. Percentage of cells harboring positive or negative level of superoxide from C6 (GFP+) or NIH3T3 (GFP−) population are recorded by flow cytometry (D) and presented as table of summary (E). The trend of population with active superoxide are graphed at (F). Images shows the active superoxide (red) are highly correlated with C6 (GFP) population and produced by NIH3T3 cell (no fluorescent label) at 345 μM or 460 μM condition as indicated by white arrows.

FIGS. 3A-3G show that Coenzyme Q10 induces differential effects on redox vulnerabilities between non-tumor and glioblastoma cells in co-culture human cell experiments. (A) Strategy of human co-culture experiments. In the human cell model, NHA (non-label) and U251 (GFP-labelled) cells are co-cultured in designated cell density. (B) Phase and fluorescent images of U251/NHA co-cultures treated with 0 or 460 μM Coenzyme Q10 demonstrating decreasing glioma cells with a relative sparing of NHA. (C) Flow cytometric analysis (scatter plot) of GFP and superoxide for U251/NHA cocultures. Cell populations are characterized based by GFP intensity (GFP-negative, GFP-low, and GFP-high). Note the increased percentage of GFP-low relative to GFP-high cells with increasing dose. (D) Quantification of cell percentages from (C) illustrating the increased number of GFP-low cells with increased dose. (E) Flow cytometric analysis of superoxide and DAPI for NHA-U251/NHA co-cultures treated with 0, 230, or 460 μM Coenzyme Q10. There is an increase in superoxide values for both GFP-low and GFP-high cells, while insignificant changes are noted for GFP-negative (NHA) cells (values depicted graphically in panel F). In contrast, DAPI levels are only significantly elevated in GFP-low cells, consistent with this population representing dying cells (depicted in Panel G).

FIG. 4 shows a patient with progressive disease following surgery confirming recurrence then 2 months on Coenzyme Q10. However, FDG-PET demonstrated decreased glucose uptake in nearly the entire involved area.

FIG. 5 shows that Coenzyme Q10 synergizes with radiation therapy (RT) to improve survival in an implanted C6 glioma rat model.

### DETAILED DESCRIPTION AND PREFERRED EMBODIMENTS

### I. Definitions

The terms “administer”, “administering” or “administration” include any method of delivery of a pharmaceutical composition or agent into a subject's system or to a particular region in or on a subject. In certain embodiments, the agent is delivered orally. In certain embodiments, the agent is administered parenterally. In certain embodiments, the agent is delivered by injection or infusion. In certain embodiments, the agent is delivered topically including transmucosally. In certain embodiments, the agent is delivered by inhalation. In certain embodiments of the invention, an agent is administered by parenteral delivery, including, intravenous, intramuscular, subcutaneous, intramedullary injections, as well as intrathecal, direct intraventricular, intraperitoneal, intranasal, or intraocular injections. In one embodiment, the compositions provided herein may be administered by injecting directly to a tumor. In some embodiments, the formulations of the invention may be administered by intravenous injection or intravenous infusion. In certain embodiments, the formulation of the invention can be administered by continuous infusion. In certain embodiments, administration is not oral. In certain embodiments, administration is systemic. In certain embodiments, administration is local. In some embodiments, one or more routes of administration may be combined, such as, for example, intravenous and intratumoral, or intravenous and peroral, or intravenous and oral, intravenous and topical, or intravenous and transdermal or transmucosal. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, etc.

“Adverse events” or “AEs” are characterized by grade depending on the severity. Some AE (e.g., nausea, low blood counts, pain, reduced blood clotting) can be treated so that the specific chemotherapeutic regimen can be continued or resumed. Some adverse events (e.g., loss of cardiac, liver, or kidney function; nausea) may not be treatable, requiring termination of treatment with the drug. Determination of AE grade and appropriate interventions can be determined by those of skill in the art. Common Terminology Criteria for Adverse Events v4.0 (CTCAE) (Publish Date: May 28, 2009) provide a grading scale for adverse events as follows:
Grade 1 Mild; asymptomatic or mild symptoms; clinical or diagnostic observations only; intervention not indicated.
Grade 2 Moderate; minimal, local or noninvasive intervention indicated; limiting age-appropriate instrumental activities of daily life (ADL).
Grade 3 Severe or medically significant but not immediately life-threatening; hospitalization or prolongation of hospitalization indicated; disabling, limiting self care ADL.
Grade 4 Life-threatening consequences; urgent intervention indicated.
Grade 5 Death related to adverse event.

An “anti-cancer agent” is understood as a drug used for the treatment of cancer. Anti-cancer agents include, but are not limited to, small molecules, hormones and hormone analogs, and biologics (e.g., antibodies, peptide drugs, nucleic acid drugs).

A “cancer therapeutic regimen” is a clinically accepted dosing protocol for the treatment of cancer that includes administration of one or more anti-cancer agents to a subject in specific amounts on a specific schedule.

The terms “cancer” or “tumor” are well known in the art and refer to the presence, e.g., in a subject, of cells possessing characteristics typical of cancer-causing cells, such as uncontrolled proliferation, immortality, metastatic potential, rapid growth and proliferation rate, decreased cell death/apoptosis, and certain characteristic morphological features.

As used herein, “co-administration” or “combination therapy” is understood as administration of two or more active agents using separate formulations or a single pharmaceutical formulation, or consecutive administration in any order such that, there is a time period while both (or all) active agents simultaneously exert their biological activities. Co-administration does not require that the agents are administered at the same time, at the same frequency, or by the same route of administration. As used herein, “co-administration” or “combination therapy” includes administration of a composition comprising a Coenzyme Q10 compound with one or more additional anti-cancer agents, e.g., chemotherapeutic agents, or administration of two or more CoQ10 compounds. Examples of anticancer agents, including chemotherapeutic agents, are provided herein.

As used herein, “continuous infusion” or “continuous intravenous infusion” is understood as administration of a dose of the formulation continuously for at least 24 hours. Continuous administration is typically facilitated by use of a pump, either an implantable or external pump. A formulation can be administered by continuous infusion in multiple, separated doses, with a break of one or more days between continuous infusion doses. A “dose” of a composition administered by continuous intravenous infusion refers to a single continuous administration of the composition to a subject. For example, administration of a composition to a subject by continuous intravenous infusion for about 24, 48, 72, 96, 120 or 144 hours would be considered a single dose of the composition. Administration of a composition to a subject by continuous intravenous infusion (e.g. for about 24, 48, 72, 96, 120 or 144 hours) followed by a break of one or more days, and then subsequently followed by a second continuous intravenous infusion (e.g. for about 24, 48, 72, 96, 120 or 144 hours), would be considered two doses of the composition.

It is understood that continuous intravenous infusion can include short interruptions of administration, for example, to change the reservoir of coenzyme Q10 being administered. For example, two 48 hour continuous intravenous infusions administered sequentially or four 24 hour continuous intravenous infusions and the like, administered without a significant pause by design (less than 4 hours, preferably less than 2 hours, preferably less than one hour, preferably about 30 minutes) between the end of one infusion and the start of the next is considered to be the same as one 96 hour continuous administration. Similarly, two 72 hour continuous intravenous infusions administered sequentially without a significant pause (e.g. less than 4 hours, preferably less than 2 hours, preferably less than one hour, preferably about 30 minutes) between the end of one infusion and the start of the next is considered to be the same as one 144 hour (6 day) continuous intravenous infusion.

As used herein, a “formulation” is understood as an active ingredient, e.g., CoQ10, a metabolite of CoQ10, a biosynthetic precursor of CoQ10, or a CoQ10 related compound, in combination with any pharmaceutically acceptable carrier. Formulations can include, but are not limited to, aqueous formulations, liposomal formulations, suspensions, emulsions, microemulsions, nanoemulsions, nanosuspensions, formulations for specific routes of administration, such as cream, lotion, and ointment formulations for topical administration, solid formulations for oral administration, and liquid formulations for injection or inhalation.

A “glioma” is a type of tumor that starts in the brain or spine and arises from glial cells (e.g. astrocytes). The most common site of gliomas is the brain. Gliomas make up about 30% of all brain and central nervous system tumors and 80% of all malignant brain tumors. Low-grade gliomas (WHO grade II) are well-differentiated (not anaplastic) and tend to exhibit benign tendencies and portend a better prognosis for the patient. High-grade [WHO grades III-IV]gliomas are undifferentiated or anaplastic; these are malignant and carry a worse prognosis.

Types of gliomas include, but are not limited to, ependymomas, astrocytomas (e.g. anaplastic astrocytomas, glioblastomas), oligodendrogliomas (e.g. anaplastic oligodendrogliomas), brainstem gliomas, optic nerve gliomas, and mixed gliomas (e.g. oligoastrocytomas) which contain cells from different types of glia. In some embodiments the glioma is non-malignant, i.e., benign. In some embodiments, the glioma is a malignant glioma.

In some embodiments, the malignant glioma is a malignant astrocytoma. In some embodiments, the malignant astrocytoma is an anaplastic astrocytoma. Anaplastic astrocytoma is a rare, malignant brain tumor that arises from astrocytes, a type of glial cell that are supportive cells in the nervous system. Normally, astrocytes are responsible for a variety of roles, including providing nutrients to neurons, maintaining the blood-brain barrier, and modulating neurotransmission. Anaplastic astrocytomas often develop in the cerebral hemispheres of the brain, but may occur in almost any area of the central nervous system. An anaplastic astrocytoma is a grade III or high-grade tumor that demonstrates focal or dispersed anaplasia (abnormal, irregular shape) cells and an increased growth index compared to grade I and II astrocytoma. The pathological diagnosis is based on appearance of cells (nuclear atypia) and growth rate (mitotic activity).

In some embodiments, the malignant astrocytoma is a glioblastoma (GBM). Glioblastoma is a grade IV glioma tumor. It is the most malignant form of astrocytoma. The features under the microscope that distinguish glioblastoma from all other grades is the presence of necrosis (dead cells) and the increase of abnormal growth of blood vessels around the tumor. Grade IV tumors are always rapidly growing and highly malignant tumors. Glioblastoma is also known as glioblastoma multiforme and grade IV astrocytoma, and is the most common and aggressive of adult primary brain tumors. While only rarely metastatic to sites outside the central nervous system (CNS), it is locally aggressive and treatment resistant. Furthermore, despite decades of study using scores of treatments and strategies, it remains incurable and there has been at best only incremental gains in survival. Less than 50% of patients survive 2 years (y) post diagnosis with the current established treatment framework of fractionated radiation therapy (RT) to a total dose of 60 Gy and concurrent temozolomide (TMZ) chemotherapy (75 mg/m2×42 days [d]) followed by 6 months (mo) of additional TMZ.

In some embodiments, the glioblastoma is a gliosarcoma. Gliosarcoma (GS) is a rare histopathological variant of isocitrate dehydrogenase (IDH)-wildtype glioblastoma characterized by a biphasic growth pattern consisting of both glial and sarcomatous components. Histologically, GS tumors are characterized by a biphasic growth pattern consisting of both glial components and areas of sarcomatous, mesenchymal differentiation often resembling fibrosarcoma.

Gliomas may be characterized as low-grade gliomas (grade I or grade II) or high-grade gliomas (grade III or grade IV). Low-grade gliomas are well-differentiated (not anaplastic) and tend to exhibit benign tendencies and portend a better prognosis for the patient. However, they have a uniform rate of recurrence and increase in grade over time so should be classified as malignant. High-grade gliomas are undifferentiated or anaplastic, malignant, and carry a worse prognosis.

The term “gray (Gy)” as used herein refers to a derived metric (SI) measurement unit of absorbed radiation dose of ionizing radiation, e.g. X-rays. The gray is defined as the absorption of one joule of ionizing radiation by one kilogram (1 J/kg) of matter, e.g. human tissue. The term “centigray (cGy)” as used herein refers to one hundredth of a gray (0.01 Gy).

As used herein, a “pharmaceutically acceptable” component is one that is suitable for use with humans and/or animals without undue adverse side effects (such as toxicity, irritation, and allergic response) commensurate with a reasonable benefit/risk ratio.

A “solid tumor” is a tumor that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient. The tumor does not need to have measurable dimensions.

Specific criteria for the staging of cancer (e.g. glioma) are dependent on the specific cancer type based on tumor size, histological characteristics, tumor markers, and other criteria known by those of skill in the art. Generally, cancer stages can be described as follows:
Stage 0 Carcinoma in situ
Stage I, Stage II, and Stage III Higher numbers indicate more extensive disease: Larger tumor size and/or spread of the cancer beyond the organ in which it first developed to nearby lymph nodes and/or tissues or organs adjacent to the location of the primary tumor
Stage IV The cancer has spread to distant tissues or organs

As used herein, the terms “treat,” “treating” or “treatment” refer, preferably, to an action to obtain a beneficial or desired clinical result including, but not limited to, alleviation or amelioration of one or more signs or symptoms of a disease or condition (e.g., regression, partial or complete), diminishing the extent of disease, stability (i.e., not worsening, achieving stable disease) state of disease, amelioration or palliation of the disease state, diminishing rate of or time to progression, and remission (whether partial or total). “Treatment” of a glioma (e.g. glioblastoma) can also mean prolonging survival as compared to expected survival in the absence of treatment. Treatment need not be curative. In certain embodiments, treatment includes one or more of a decrease in pain or an increase in the quality of life (QOL) as judged by a qualified individual, e.g., a treating physician, e.g., using accepted assessment tools of pain and QOL. In certain embodiments, treatment does not include one or more of a decrease in pain or an increase in the quality of life (QOL) as judged by a qualified individual, e.g., a treating physician, e.g., using accepted assessment tools of pain and QOL.

RECIST criteria are clinically accepted assessment criteria used to provide a standard approach to solid tumor measurement and provide definitions for objective assessment of change in tumor size for use in clinical trials. Such criteria can also be used to monitor response of an individual undergoing treatment for a solid tumor. The RECIST 1.1 criteria are discussed in detail in Eisenhauer et al., New response evaluation criteria in solid tumors: Revised RECIST guideline (version 1.1). Eur. J. Cancer. 45:228-247, 2009, which is incorporated herein by reference. Response criteria for target lesions include:
Complete Response (CR): Disappearance of all target lesions. Any pathological lymph nodes (whether target or non-target) must have a reduction in short axis to <10 mm.
Partial Response (PR): At least a 30% decrease in the sum of diameters of target lesion, taking as a reference the baseline sum diameters.
Progressive Diseases (PD): At least a 20% increase in the sum of diameters of target lesions, taking as a reference the smallest sum on the study (this includes the baseline sum if that is the smallest on the study). In addition to the relative increase of 20%, the sum must also demonstrate an absolute increase of at least 5 mm. (Note: the appearance of one or more new lesions is also considered progression.)
Stable Disease (SD): Neither sufficient shrinkage to qualify for PR nor sufficient increase to qualify for PD, taking as a reference the smallest sum diameters while on study.

RECIST 1.1 criteria also consider non-target lesions which are defined as lesions that may be measureable, but need not be measured, and should only be assessed qualitatively at the desired time points. Response criteria for non-target lesions include:
Complete Response (CR): Disappearance of all non-target lesions and normalization of tumor marker levels. All lymph nodes must be non-pathological in size (<10 mm short axis).
Non-CR/Non-PD: Persistence of one or more non-target lesion(s) and/or maintenance of tumor marker level above the normal limits.
Progressive Disease (PD): Unequivocal progression (emphasis in original) of existing non-target lesions. The appearance of one or more new lesions is also considered progression. To achieve “unequivocal progression” on the basis of non-target disease, there must be an overall level of substantial worsening of non-target disease such that, even in the presence of SD or PR in target disease, the overall tumor burden has increased sufficiently to merit discontinuation of therapy. A modest “increase” in the size of one or more non-target lesions is usually not sufficient to qualify for unequivocal progression status. The designation of overall progression solely on the basis of change in non-target disease in the face of SD or PR in target disease will therefore be extremely rare.

A “subject who has failed treatment for the glioma” or a “subject who has failed a cancer therapeutic regimen for the glioma” is a subject with a glioma (e.g. glioblastoma) that does not respond, or ceases to respond to treatment with a cancer therapeutic regimen per RECIST 1.1 criteria (see, Eisenhauer et al., 2009 and as discussed above), i.e., does not achieve a complete response, partial response, or stable disease in the target lesion; or does not achieve complete response or non-CR/non-PD of non-target lesions, either during or after completion of the cancer therapeutic regimen, either alone or in conjunction with surgery and/or radiation therapy which, when possible, are often clinically indicated in conjunction with anti-cancer agents. A failed cancer therapeutic regime results in, e.g., tumor growth, increased tumor burden, and/or tumor metastasis. A failed cancer therapeutic regimen as used herein includes a treatment regimen that was terminated due to a dose limiting toxicity, e.g., a grade III or a grade IV toxicity that cannot be resolved to allow continuation or resumption of treatment with the cancer therapeutic agent or regimen that caused the toxicity. A failed cancer therapeutic regimen includes a treatment regimen that does not result in at least stable disease for all target and non-target lesions for an extended period, e.g., at least 1 month, at least 2 months, at least 3 months, at least 4 months, at least 5 months, at least 6 months, at least 12 months, at least 18 months, or any time period less than a clinically defined cure. A failed cancer therapeutic regimen includes a treatment regimen that results in progressive disease of at least one target lesion during treatment with the chemotherapeutic agent, or results in progressive disease less than 2 weeks, less than 1 month, less than two months, less than 3 months, less than 4 months, less than 5 months, less than 6 months, less than 12 months, or less than 18 months after the conclusion of the treatment regimen, or less than any time period less than a clinically defined cure. In a particular embodiment, the failed cancer therapeutic regimen comprises radiation therapy.

A failed cancer therapeutic regimen does not include a treatment regimen wherein the subject treated for a glioma (e.g. glioblastoma) achieves a clinically defined cure, e.g., 5 years of complete response after the end of the treatment regimen, and wherein the subject is subsequently diagnosed with a distinct cancer, e.g., more than 5 years, more than 6 years, more than 7 years, more than 8 years, more than 9 years, more than 10 years, more than 11 years, more than 12 years, more than 13 years, more than 14 years, or more than 15 years after the end of the treatment regimen. For example, a subject who suffered from a glioma (e.g. glioblastoma) may develop cancer later in life after being cured of the glioma. In such a subject, the cancer therapeutic regimen to treat the glioma is considered to have been successful.

A “refractory glioma” is a glioma (e.g. glioblastoma) which is either initially unresponsive to a cancer therapeutic regimen (e.g. radiation therapy), or which is initially responsive to a cancer therapeutic regimen (e.g. radiation therapy) but becomes unresponsive to the cancer therapeutic regimen (e.g. radiation therapy) over time.

As used herein, the term “safe and therapeutic effective amount” refers to the quantity of a component which is sufficient to yield a desired therapeutic response without undue adverse side effects (such as toxicity, irritation, or allergic response) commensurate with a reasonable benefit/risk ratio when used in the manner of this disclosure.

As used herein, the term “survival” refers to the continuation of life of a subject which has been treated for a disease or condition, e.g., a glioma (e.g. glioblastoma). The time of survival can be defined from an arbitrary point such as time of entry into a clinical trial, time from completion or failure or an earlier treatment regimen, time from diagnosis, etc.

As used herein, the term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human and may be referred to as a patient.

As used herein, the term “therapeutically effective amount” refers to an amount, e.g., of a compound of the present disclosure, effective to yield the desired therapeutic response or sufficient to treat a disease in a subject. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives. The “therapeutically effective amount” will vary depending on the compound, the disease and its severity and the age, weight, etc., of the patient to be treated. A therapeutically effective amount can be administered in one or more administrations.

The term “therapeutic effect” refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically-effective amount of a compound will depend on its therapeutic index, solubility, and the like.

The articles “a”, “an” and “the” are used herein to refer to one or to more than one (i.e. to at least one) of the grammatical object of the article unless otherwise clearly indicated by contrast. By way of example, “an element” means one element or more than one element.

The term “including” is used herein to mean, and is used interchangeably with, the phrase “including but not limited to”.

The term “or” is used herein to mean, and is used interchangeably with, the term “and/or,” unless context clearly indicates otherwise.

The term “such as” is used herein to mean, and is used interchangeably, with the phrase “such as but not limited to”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical group(s) in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### II. Coenzyme 010 Compounds

Coenzyme Q10 (CoQ10) compounds are intended to include a class of CoQ10 related compounds. Coenzyme Q10 compounds effective for the methods described herein include CoQ10, a metabolite of CoQ10, a biosynthetic precursor of CoQ10, an analog of CoQ10, a derivative of CoQ10, and CoQ10 related compounds. An analog of CoQ10 includes analogs having no or at least one isoprenyl repeats. CoQ10 has the following structure:

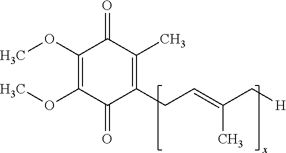

wherein x is 10. In the instant invention, CoQ10 compounds can include derivatives of CoQ10 in which x is any number of isoprenyl units from 4-10, or any number of isoprenyl units from 6-10, or any number of isoprenyl units from 8-10, or 9-10 isoprenyl units. CoQ10 includes the fully oxidized version, also known as ubiquinone, the partially oxidized version, also known as semiquinone or ubisemiquinone, or the fully reduced version, also known as ubiquinol; or any mixtures or combinations thereof. In certain embodiments, the composition comprising Coenzyme Q10 for use in the treatment methods herein (e.g., treatment of a glioma, e.g. glioblastoma) is ubiquinone or fully oxidized CoQ10. In certain embodiments, the composition comprising Coenzyme Q10 for use in the treatment methods herein (e.g., treatment of a glioma, e.g. glioblastoma) is ubiquinol.

In certain embodiments of the present invention, the therapeutic agent is Coenzyme Q10 (CoQ10). Coenzyme Q10, also referred to herein as CoQ10, is also known as ubiquinone, or ubidecarenone. CoQ10 is art-recognized and further described in International Publication No. WO 2005/069916 (Appln. No. PCT/US2005/001581), WO 2008/116135 (Appln. No. PCT/US08/57786), WO2010/132507 (Appln. No. PCT/US2010/034453), WO 2011/112900 (Appln. No. PCT/US2011/028042), and WO2012/174559 (Appln. No. PCT/US2012/043001) the entire contents of each of which are expressly incorporated by reference herein. CoQ10 is one of a series of polyprenyl 2,3-dimethoxy-5-methylbenzoquinone (ubiquinone) present in the mitochondrial electron transport systems of eukaryotic cells. Human cells produce CoQ10 exclusively and it is found in cell and mitochondrial membranes of all human cells, with the highest levels in organs with high energy requirements, such as the liver and the heart. The body pool of CoQ10 has been estimated to be about 2 grams, of which more than 50% is endogenous. Approximately 0.5 grams of CoQ10 is required from the diet or biosynthesis each day. CoQ10 is produced in ton quantities from the worldwide supplement market and can be obtained from Kaneka, with plants in Pasadena, Texas and Takasagoshi, Japan.

Coenzyme Q10 related compounds include, but are not limited to, benzoquinones, isoprenoids, farnesols, farnesyl acetate, farnesyl pyrophosphate, 1-phenylalanine, d-phenylalanine, dl-phenylalanine, 1-tyrosine, d-tyrosine, dl-tyrosine, 4-hydroxy-phenylpyruvate, 4-hydroxy-phenyllactate, 4-hydroxy-cinnamate, dipeptides and tripeptides of tyrosine or phenylalanine, 3,4-dihydroxymandelate, 3-methoxy-4-hydroxyphenylglycol, 3-methoxy-4-hydroxymandelate, vanillic acid, phenylacetate, pyridoxine, S-adenosyl methionine, panthenol, mevalonic acid, isopentyl pyrophosphate, phenylbutyrate, 4-hydroxy-benzoate, decaprenyl pyrophosphate, beta-hydroxybutyrate, 3-hydroxy-3-methyl-glutarate, acetylcarnitine, acetoacetylcarnitine, acetylglycine, acetoacetylglycine, carnitine, acetic acid, pyruvic acid, 3-hydroxy-3-methylglutarylcarnitine, all isomeric forms of serine, alanine, cysteine, glycine, threonine, hydroxyproline, lysine, isoleucine, and leucine, even carbon number C₄ to C₈ fatty acids (butyric, caproic, caprylic, capric, lauric, myristic, palmitic, and stearic acids) salts of carnitine and glycine, e.g., palmitoylcarnitine and palmitoylglycine, and 4-hydroxy-benzoate polyprenyltransferase, any salts of these compounds, as well as any combinations thereof, and the like. In certain embodiments, such agents can be used for the treatment of certain cancers as provided herein, e.g, a glioma (e.g. glioblastoma), according to the methods provided herein.

Metabolites and biosynthetic precursors of CoQ10 include, but are not limited to, those compounds that are formed between the chemical/biological conversion of tyrosine and acetyl-CoA to ubiquinol. Intermediates of the coenzyme biosynthesis pathway include tyrosine, acetyl-CoA, 3-hexaprenyl-4-hydroxybenzoate, 3-hexaprenyl-4,5-dihydroxybenzoate, 3-hexaprenyl-4-hydroxy-5-methoxybenzoate, 2-hexaprenyl-6-methoxy-1,4-benzoquinone, 2-hexaprenyl-3-methyl-6-methoxy-1,4-benzoquinone, 2-hexaprenyl-3-methyl-5-hydroxy-6-methoxy-1,4-benzoquinone, 3-Octaprenyl-4-hydroxybenzoate, 2-octaprenylphenol, 2-octaprenyl-6-metholxyphenol, 2-octaprenyl-3-methyl-6-methoxy-1,4-benzoquinone, 2-octaprenyl-3-methyl-5-hydroxy-6-methoxy-1,4-benzoquinone, 2-decaprenyl-3-methyl-5-hydroxy-6-methoxy-1,4-benzoquinone, 2-decaprenyl-3-methyl-6-methoxy-1,4-benzoquinone, 2-decaprenyl-6-methoxy-1,4-benzoquinone, 2-decaprenyl-6-methoxyphenol, 3-decaprenyl-4-hydroxy-5-methoxybenzoate, 3-decaprenyl-4,5-dihydroxybenzoate, 3-decaprenyl-4-hydroxybenzoate, 4-hydroxy phenylpyruvate, 4-hydroxyphenyllactate, 4-hydroxy-benzoate, 4-hydroxycinnamate, and hexaprenydiphosphate. In certain embodiments, such agents can be used for the treatment of certain cancers as provided herein, e.g, a glioma (e.g. glioblastoma), according to the methods provided herein.

### III. Compositions

The present disclosure provides compositions comprising a Coenzyme Q10 compound for the treatment of gliomas (e.g. glioblastomas). The compositions of the present disclosure can be administered to a patient either by themselves, or in pharmaceutical compositions where it is mixed with suitable carriers or excipient(s). In treating a patient exhibiting a disorder of interest, e.g., glioma, e.g. glioblastoma, a therapeutically effective amount of the composition comprising a Coenzyme Q10 compound is administered. A therapeutically effective dose refers to that amount of the compound which results in at least stable disease or a prolongation of survival in a patient.

Suitable routes of administration of the present compositions of the invention may include parenteral delivery, including, intravenous, intramuscular, subcutaneous, intramedullary injections, as well as intrathecal, direct intraventricular, intraperitoneal, intranasal, or intraocular injections, just to name a few. In one embodiment, the compositions provided herein may be administered by injecting directly to a tumor. In some embodiments, the formulations of the invention may be administered by intravenous injection or intravenous infusion. In some embodiments, the formulation is administered by continuous infusion. In one embodiment, the compositions of the invention are administered by intravenous injection. In one embodiment, the compositions of the invention are administered by intravenous infusion. In one embodiment, the Coenzyme Q10 is administered by continuous intravenous infusion. In some embodiments, the Coenzyme Q10 is administered by continuous intravenous infusion for at least 36 hours, at least 48 hours, at least 60 hours, at least 72 hours, at least 84 hours, at least 96 hours, at least 108 hours, at least 120 hours, at least 132 hours, or at least 144 hours. In some embodiments, the continuous intravenous infusion is administered for about 36 hours, about 48 hours, about 60 hours, about 72 hours, about 84 hours, about 96 hours, about 108 hours, about 120 hours, about 132 hours, or about 144 hours. In a particular embodiment, the continuous intravenous infusion is administered for at least 72 hours, or for about 72 hours (about 3 days). In a further particular embodiment, the continuous infusion is administered for at least 96 hours, or for about 96 hours (about 4 days).

Where the route of administration is, for example intravenous infusion, embodiments are provided herein where the IV infusion comprises the active agent, e.g., CoQ10, at approximately a 40 mg/mL concentration. Where the composition is administered by IV infusion, it can be diluted in a pharmaceutically acceptable aqueous solution such as phosphate buffered saline or normal saline. In some embodiments, one or more routes of administration may be combined, such as, for example, intravenous and intratumoral, or intravenous and peroral, or intravenous and oral, or intravenous and topical, transdermal, or transmucosal.

In some embodiments, suitable routes of administration of the present compositions of the invention include topical, inhalable, or oral administration.

The compositions described herein may be administered to a subject in any suitable formulation. These include, for example, liquid, semi-solid, and solid dosage forms, such as liquid solutions (e.g., injectable and infusible solutions), dispersions or suspensions, tablets, pills, powders, creams, lotions, liniments, ointments, or pastes, drops for administration to the eye, ear or nose, liposomes, and suppositories. The preferred form depends on the intended mode of administration and therapeutic application.

In certain embodiments, a composition comprising a Coenzyme Q10 compound may be prepared with a carrier that will protect against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

For example, a composition comprising a Coenzyme Q10 compound can be formulated for parenteral delivery, e.g., for subcutaneous, intravenous, intramuscular, or intratumoral injection. The compositions may be administered in a single bolus, multiple injections, or by continuous infusion (for example, intravenously or by peritoneal dialysis). For parenteral administration, the compositions may be formulated in a sterilized pyrogen-free form.

Use of pharmaceutically acceptable carriers to formulate the Coenzyme Q10 compounds disclosed herein, for the practice of the present invention, into dosages suitable for systemic administration is within the scope of the present disclosure. With proper choice of carrier and suitable manufacturing practice, the compositions of the present disclosure, in particular, those formulated as solutions, may be administered parenterally, such as by intravenous injection.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices may be desirable. The data obtained from these cell culture assays and animal studies can be used in formulating a range of dosage for use in human. The dosage of such compounds may be within a range of circulating concentrations that include the ED50 with little or no toxicity. The dosage may vary within this range depending upon the dosage form employed and the route of administration utilized.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers including excipients and auxiliaries which facilitate processing of the active compounds into preparations which can be used pharmaceutically. The preparations formulated for intravenous administration may be in the form of solutions of colloidal dispersion.

Pharmaceutical compositions for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

### IV. Formulations

The active agent, e.g., a CoQ10 compound, can be delivered in any pharmaceutically acceptable carrier for the desired route of administration. As used herein, formulations including CoQ10 compounds are formulated for any route of administration unless otherwise clearly indicated. In preferred embodiments, the formulations are for administration by injection, infusion, or topical administration. In certain embodiments, the CoQ10 compounds are not delivered orally.

Preferred therapeutic formulations for use in the methods of the invention comprise the active agent (e.g., a CoQ10 compound) in a microparticle formation, e.g., for intravenous administration. Such intravenous formulations are provided, for example, in WO2011/112900 (Appln. No. PCT/US2011/028042), the entire contents of which are expressly incorporated herein by reference, and an exemplary intravenous formulation as described in WO2011/112900 (Appln. No. PCT/US2011/028042). Through high pressure homogenization, active agent (e.g., a CoQ10 compound) particles are reduced to produce particles that are small enough to pass through a 200-nm sterilizing filter. Particles that are small enough to pass through a 200-nm sterilizing filter can be injected intravenously. These particles are much smaller than blood cells and therefore will not embolize capillaries. Red blood cells for example are 6-micron×2-micron disks. The particles are dispersed to and are encased or surrounded by a stabilizing agent. While not wishing to be bound by any theory, it is believed that the stabilizing agents are attracted to the hydrophobic therapeutic agent such that the dispersed particles of the hydrophobic therapeutic agent are surrounded by the stabilizing agent forming a suspension or an emulsion. The dispersed particles in the suspension or emulsion comprises a stabilizing agent surface and a core consisting of the hydrophobic therapeutic agent, e.g., a CoQ10 compound, in a solid particulate form (suspension) or in an immiscible liquid form (emulsion). The dispersed particles can be entrenched in the lipophilic regions of a liposome.

Dispersed colloidal systems permit a high drug load in the formulation without the use of co-solvents. Additionally, high and relatively reproducible plasma levels are achieved without the dependence on endogenous low-density lipoprotein carriers. More importantly, the formulations allow sustained high drug levels in solid tumors due to the passive accumulation of the colloidal particles of the hydrophobic therapeutic agent.

A preferred intravenous formulation substantially comprises a continuous phase of water and dispersed solids (suspension) or dispersed immiscible liquid (emulsion). Dispersed colloidal systems, in which the particles are composed largely of the active agent (drug) itself, can often deliver more drug per unit volume than continuous solubilizing systems, if the system can be made adequately stable.

As the formulation medium, the aqueous solution may include Hank's solution, Ringer's solution, phosphate buffered saline (PBS), physiological saline buffer or other suitable salts or combinations to achieve the appropriate pH and osmolarity for parenterally delivered formulations. Aqueous solutions can be used to dilute the formulations for administration to the desired concentration. For example, aqueous solutions can be used to dilute a formulation for intravenous administration from a concentration of about 4% w/v to a lower concentration to facilitate administration of lower doses of CoQ10. The aqueous solution may contain substances which increase the viscosity of the solution, such as sodium carboxymethyl cellulose, sorbitol, or dextran.

The active agent (e.g., a CoQ10 compound) is dispersed in the aqueous solution such that a colloidal dispersion is formed wherein the nano-dispersion particles of the hydrophobic therapeutic agent are covered or encased or encircled by the dispersion stabilizing agents to form nano-dispersions of the active agent (e.g., a CoQ10 compound) particles. The nano-dispersed active agent (e.g., a CoQ10 compound) particles have a core formed of the hydrophobic therapeutic agent that is surrounded by the stabilizing agent. Similarly, in certain aspects, the stabilizing agent is a phospholipid having both a hydrophilic and lipophilic portion. The phospholipids form liposomes or other nanoparticles upon homogenization. In certain aspects these liposomes are bi-layered unilamellar liposomes while in other embodiments the liposomes are bi-layered multi-lamellar liposomes. The dispersed active agent (e.g., a CoQ10 compound) particles are dispersed in the lipophilic portion of the bi-layered structure of the liposome formed from the phospholipids. In certain other aspects the core of the liposome, like the core of the nano-dispersion of active agent (e.g., a CoQ10 compound) particles, is formed of the hydrophobic therapeutic agent and the outer layer is formed of the bi-layered structure of the phospholipid. In certain embodiments the colloidal dispersions are treated by a lyophilization process whereby the nanoparticle dispersion is converted to a dry powder.

In some embodiments, the formulation for injection or infusion used is a 4% sterile aqueous colloidal dispersion containing CoQ10 in a nanosuspension as prepared in WO2011/112900, the entire contents of which are incorporated herein by reference. In certain embodiments, the formulation includes an aqueous solution; a hydrophobic active agent, e.g., CoQ10, a CoQ10 precursor or metabolite or a CoQ10 related compound, dispersed to form a colloidal nano-dispersion of particles; and at least one of a dispersion stabilizing agent and an opsonization reducer; wherein the colloidal nano-dispersion of the active agent is dispersed into nano-dispersion particles having a mean size of less than 200-nm.

In certain embodiments, the dispersion stabilizing agent includes, but is not limited to, pegylated castor oil, Cremphor® EL, Cremophor® RH 40, Pegylated vitamin E, Vitamin E TPGS, and Dimyristoylphosphatidyl choline (DMPC).

In certain embodiments, the opsonization reducer is a poloxamer or a poloxamines.

In certain embodiments, the colloidal nano-dispersion is a suspension or an emulsion. Optionally, a colloidal nano-dispersion is in a crystalline form or a super-cooled melt form.

In certain embodiments, the formulation for injection or infusion includes a lyoprotectant such as a nutritive sugar including, but not limited to, lactose, mannose, maltose, galactose, fructose, sorbose, raffinose, neuraminic acid, glucosamine, galactosamine, N-methylglucosamine, mannitol, sorbitol, arginine, glycine and sucrose, or any combination thereof.

In certain embodiments, the formulation for injection or infusion includes an aqueous solution; a hydrophobic active agent dispersed to form a colloidal nano-dispersion of particles; and at least one of a dispersion stabilizing agent and an opsonization reducer. The colloidal nano-dispersion of the active agent is dispersed into nano-dispersion particles having sizes of less than 200-nm. In some embodiments the dispersion stabilizing agent is selected from natural or semisynthetic phospholipids. For example, suitable stabilizing agents include polyethoxylated (a/k/a pegylated) castor oil (Cremophor® EL), polyethoxylated hydrogenated castor oil (Cremophor® RH 40), Tocopherol polyethylene glycol succinate (Pegylated vitamin E, Vitamin E TPGS), Sorbitan fatty acid esters (Spans®), Bile acids and bile-acid salts or Dimyristoylphosphatidyl choline (DMPC). In some embodiments the stabilizing agent is DMPC.

In certain embodiments the formulation is suitable for parenteral administration, including intravenous, intraperitoneal, orthotopical, intracranial, intramuscular, subcutaneous, intramedullary injections, as well as intrathecal, direct intraventricular, intranasal, or intraocular injections. In certain embodiments, the formulation contains CoQ10, dimyristoyl-phophatidylcholine, and poloxamer 188 in a ratio of 4:3:1.5 respectively that is designed to stabilize the nanosuspension of the particles. In some embodiments, the formulation includes a phosphate buffer saline solution which contains sodium phosphate dibasic, potassium phosphate monobasic, potassium chloride, sodium chloride and water for injection. In certain embodiments, the 4% sterile aqueous colloidal dispersion containing CoQ10 in a nanosuspension is diluted in the phosphate buffered saline solution provided, e.g., 1:1, 1:2, 1:3, 1:4. 1:5, 1:6, 1:7, 1:8. 1:9, 1:10, 1:11, 1:12, 1:13, 1:14. 1:15, 1:16, 1:17, 1:18. 1:19, 1:20, or other appropriate ratio bracketed by any two of the values.

In some embodiments, the formulation is a topical formulation. Topical formulations of CoQ10 compounds are provided, for example in WO2010/132507 (PCT Appln. No. PCT/US2010/034453), WO2008116135 (PCT Appln. No. PCT/US2008/116135), and WO2005/069916 (PCT Appln. PC/US2005/001581), the entire contents of each of which are expressly incorporated herein by reference.

Formulations suitable for topical administration include liquid or semi-liquid preparations suitable for penetration through the skin, such as liniments, lotions, creams, ointments or pastes, and drops suitable for administration to the eye, ear, or nose. Drops according to the present disclosure may include sterile aqueous or oily solutions or suspensions and may be prepared by dissolving the active ingredient in a suitable aqueous solution of a bactericidal and/or fungicidal agent and/or any other suitable preservative, and in some embodiments including a surface active agent. The resulting solution may then be clarified and sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the present disclosure include those suitable for application to the skin or eye. An eye lotion may include a sterile aqueous solution optionally containing a bactericide and may be prepared by methods similar to those for the preparation of drops. Lotions or liniments for application to the skin may also include an agent to hasten drying and to cool the skin, such as an alcohol, and/or a moisturizer such as glycerol or an oil such as castor oil or arachis oil.

Creams, ointments or pastes useful in the methods of the invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy basis. The basis may include hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogels. The formulation may incorporate any suitable surface active agent such as an anionic, cationic or non-ionic surface active such as sorbitan esters or polyoxyethylene derivatives thereof. Suspending agents such as natural gums, cellulose derivatives or inorganic materials such as silicaceous silicas, and other ingredients such as lanolin, may also be included.

In some embodiments, the remaining component of a topical delivery vehicle may be water or a water phase, in embodiments purified, e.g. deionized, water, glycerine, propylene glycol, ethoxydiglycol, phenoxyethanol, and cross linked acrylic acid polymers. Such delivery vehicle compositions may contain water or a water phase in an amount of from about 50 to about 95 percent, based on the total weight of the composition. The specific amount of water present is not critical, however, being adjustable to obtain the desired viscosity (usually about 50 cps to about 10,000 cps) and/or concentration of the other components. The topical delivery vehicle may have a viscosity of at least about 30 centipoises.

Topical formulations can also include an oil phase including, for example, oil phase which, in turn, may include emollients, fatty alcohols, emulsifiers, combinations thereof, and the like. For example, an oil phase could include emollients such as C12-15 alkyl benzoates (commercially available as FINSOLV™ TN from Finetex Inc. (Edison, N.J.)), capric-caprylic triglycerides (commercially available from Huls as MIGLYOL™ 812), and the like. Other suitable emollients which may be utilized include vegetable derived oils (corn oil, safflower oil, olive oil, macadamian nut oil, etc.); various synthetic esters, including caprates, linoleates, dilinoleates, isostearates, fumarates, sebacates, lactates, citrates, stearates, palmitates, and the like; synthetic medium chain triglycerides, silicone oils or polymers; fatty alcohols such as cetyl alcohol, stearyl alcohol, cetearyl alcohol, lauryl alcohol, combinations thereof, and the like; and emulsifiers including glyceryl stearate, PEG-100 stearate, Glyceryl Stearate, Glyceryl Stearate SE, neutralized or partially neutralized fatty acids, including stearic, palmitic, oleic, and the like; vegetable oil extracts containing fatty acids, Ceteareth®-20, Ceteth®-20, PEG-150 Stearate, PEG-8 Laurate, PEG-8 Oleate, PEG-8 Stearate, PEG-20 Stearate, PEG-40 Stearate, PEG-150 Distearate, PEG-8 Distearate, combinations thereof, and the like; or other non-polar cosmetic or pharmaceutically acceptable materials used for skin emolliency within the purview of those skilled in the art, combinations thereof, and the like.

Topical formulations can also include a liposomal concentrate including, for example, a phospholipid such as lecithin, lysolecithin, phosphatidylcholine, phosphatidylethanolamine, phosphatidylinositol, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, and combinations thereof, at least one lipophilic bioactive agent, and at least one solubilizer. The liposomal concentrate may be in combination with at least one pharmaceutically acceptable carrier possessing at least one permeation enhancer in an amount from about 0.5% by weight to about 20% by weight of the composition. The phospholipid may present in the composition in an amount from about 2% to about 20% by weight of the composition and the bioactive agent may be present in an amount from about 0.5% to about 20% by weight of the composition.

Transdermal skin penetration enhancers can also be used to facilitate delivery of CoQ10. Illustrative are sulfoxides such as ethoxydiglycol, 1,3-butylene glycol, isopentyl diol, 1,2-pentane diol, propylene glycol, 2-methyl propan-2-ol, propan-2-ol, ethyl-2-hydroxypropanoate, hexan-2,5-diol, di(2-hydroxypropyl)ether, pentan-2,4-diol, acetone, polyoxyethylene(2)methyl ether, 2-hydroxypropionic acid, 2-hydroxyoctanoic acid, propan-1-ol, 1,4 dioxane, tetrahydrofuran, butan-1,4-diol, propylene glycol dipelargonate, polyoxypropylene 15 stearyl ether, octyl alcohol, polyoxyethylene ester of oleyl alcohol, oleyl alcohol, lauryl alcohol, dioctyl adipate, dicapryl adipate, diisopropyl adipate, diisopropyl sebacate, dibutyl sebacate, diethyl sebacate, dimethyl sebacate, dioctyl sebacate, dibuyl suberate, dioctyl azelate, dibenzyl sebacate, dibutyl phthalate, dibutyl azelate, ethyl myristate, dimethyl azelate, butyl myristate, dibutyl succinate, didecyl phthalate, decyl oleate, ethyl caproate, ethyl salicylate, isopropyl palmitate, ethyl laurate, 2-ethyl-hexyl pelargonate, isopropyl isostearate, butyl laurate, benzyl benzoate, butyl benzoate, hexyl laurate, ethyl caprate, ethyl caprylate, butyl stearate, benzyl salicylate, 2-hyroxyoctanoic acid, dimethyl sulphoxide, methyl sufonyl methane, n,n-dimethyl acetamide, n,n-dimethyl formamide, 2-pyrrolidone, 1-methyl-2-pyrrolidone, 5-methyl-2-pyrrolidone, 1,5-dimethyl-2-pyrrolidone, 1-ethyl-2-pyrrolidone, phosphine oxides, sugar esters, tetrahydrofurfural alcohol, urea, diethyl-m-toluamide, 1-dodecylazacyloheptan-2-one, and combinations thereof.

Solubilizers, particularly for topical administration can include, but are not limited to, polyoxyalkylene dextrans, fatty acid esters of saccharose, fatty alcohol ethers of oligoglucosides, fatty acid esters of glycerol, fatty acid esters of polyoxyethylenes, polyethoxylated fatty acid esters of sorbitan, fatty acid esters of poly(ethylene oxide), fatty alcohol ethers of poly(ethylene oxide), alkylphenol ethers of poly(ethylene oxide), polyoxyethylene-polyoxypropylene block copolymers, ethoxylated oils, and combinations thereof.

Topical formulations can include emollients, including, but not limited to, C12-15 alkyl benzoates, capric-caprylic triglycerides, vegetable derived oils, caprates, linoleates, dilinoleates, isostearates, fumarates, sebacates, lactates, citrates, stearates, palmitates, synthetic medium chain triglycerides, silicone oils, polymers and combinations thereof; the fatty alcohol is selected from the group consisting of cetyl alcohol, stearyl alcohol, cetearyl alcohol, lauryl alcohol and combinations thereof; and the emulsifier is selected from the group consisting of glyceryl stearate, polyethylene glycol 100 stearate, neutralized fatty acids, partially neutralized fatty acids, polyethylene glycol 150 stearate, polyethylene glycol 8 laurate, polyethylene glycol oleate, polyethylene glycol 8 stearate, polyethylene glycol 20 stearate, polyethylene glycol 40 stearate, polyethylene glycol 150 distearate, polyethylene glycol 8 distearate, and combinations thereof.

Topical formulations can include a neutralization phase comprising one or more of water, amines, sodium lactate, and lactic acid.

The water phase can further optionally include a permeation enhancer optionally in combination with a viscosity modifier selected from the group consisting of cross linked acrylic acid polymers, pullulan, mannan, scleroglucans, polyvinylpyrrolidone, polyvinyl alcohol, guar gum, hydroxypropyl guar gum, xanthan gum, acacia gum, arabia gum, tragacanth, galactan, carob gum, karaya gum, locust bean gum, carrageenin, pectin, amylopectin, agar, quince seed, rice starch, corn starch, potato starch, wheat starch, algae extract, dextran, succinoglucan, carboxymethyl starch, methylhydroxypropyl starch, sodium alginate, alginic acid propylene glycol esters, sodium polyacrylate, polyethylacrylate, polyacrylamide, polyethyleneimine, bentonite, aluminum magnesium silicate, laponite, hectonite, and anhydrous silicic acid.

Topical formulations can also include a pigment such as titanium dioxide.

In an embodiment, a topical formulation for use in the methods of the invention includes an oil phase comprising C12-15 alkyl benzoates or capric/caprylic triglyceride, cetyl alcohol, stearyl alcohol, glyceryl stearate, and polyethylene glycol 100 stearate, in an amount of from about 5% to about 20% by weight of the composition; a water phase comprising glycerin, propylene glycol, ethoxydiglycol, phenoxyethanol, water, and a crosslinked acrylic acid polymer, in an amount of from about 60 to about 80% by weight of the composition; a neutralization phase comprising water, triethanolamine, sodium lactate, and lactic acid, in an amount of from about 0.1% to about 15% by weight of the composition; a pigment comprising titanium dioxide in an amount of from about 0.2% to about 2% by weight of the composition; and a liposomal concentrate comprising a polyethoxylated fatty acid ester of sorbitan, coenzyme Q10, a phosphatidylcholine lecithin, phenoxyethanol, propylene glycol, and water, in an amount of from about 0.1% to about 30% by weight of the composition, wherein the propylene glycol and ethoxydiglycol are present in a combined amount of from 3% by weight to about 15% by weight of the composition and the coenzyme Q10 is present in an amount of from about 0.75% by weight to about 10% by weight of the composition. Other formulations for use in the methods of the invention are provided, for example, in WO2008/116135 (PCT Application No. PCT/US08/57786), and in WO2010/132507 (PCT/US2010/034453), the entire contents of each of which are expressly incorporated herein by reference.

In one embodiment, a topical formulation for use in the methods of the invention is a 3% CoQ10 cream as described in US 2011/0027247, the entire contents of which are incorporated by reference herein. In one embodiment, the 3% CoQ10 comprises: (1) a phase A having C12-15 alkyl benzoate or capric/caprylic triglyceride at about 4.0% w/w of the composition, cetyl alcohol at about 2.00% w/w of the composition, stearyl alcohol at about 1.5% w/w, glyceryl stearate and PEG-100 at about 4.5% w/w; (2) a phase B having glycerin at about 2.00% w/w, propylene glycol at about 1.5% w/w, ethoxydiglycol at about 5.0% w/w, phenoxyethanol at about 0.475% w/w, a carbomer dispersion at about 40% w/w, purified water at about 16.7% w/w; (3) a phase C having triethanolamine at about 1.3% w/w, lactic acid at about 0.5% w/w, sodium lactate solution at about 2.0% w/w, water at about 2.5% w/w; (4) a phase D having titanium dioxide at about 1.0% w/w; and (5) a phase E having CoQ10 21% concentrate at about 15.0% w/w.

A CoQ10 21% concentrate composition (phase E in above 3% cream) can be prepared by combining phases A and B as described below. Phase A includes Ubidecarenone USP (CoQ10) at 21% w/w and polysorbate 80 NF at 25% w/w. Phase B includes propylene glycol USP at 10.00% w/w, phenoxyethanol NF at 0.50% w/w, lecithin NF (PHOSPHOLIPON 85G) at 8.00% w/w and purified water USP at 35.50% w/w. All weight percentages are relative to the weight of the entire CoQ10 21% concentrate composition. The percentages and further details are listed in the following table.

TABLE 1

| Phase | Trade Name | INCI Name | Percent |
|---|---|---|---|
| A | RITABATE 80 | POLYSORBATE 80 | 25.000 |
| A | UBIDECARENONE | UBIQUINONE | 21.000 |
| B | PURIFIED WATER | WATER | 35.500 |
| B | PROPYLENE | PROPYLENE | 10.000 |
| | GLYCOL | GLYCOL | |
| B | PHENOXYETHANOL | PHENOXYETHANOL | 0.500 |
| B | PHOSPHOLIPON 85G | LECITHIN | 8.000 |
| Totals | | | 100.000 |

The phenoxyethanol and propylene glycol are placed in a suitable container and mixed until clear. The required amount of water is added to a second container (Mix Tank 1). Mix Tank 1 is heated to between 45 and 55° C. while being mixed. The phenoxyethanol/propylene glycol solution is added to the water and mixed until it was clear and uniform. When the contents of the water phase in Mix Tank 1 are within the range of 45 to 55° C., Phospholipon G is added with low to moderate mixing. While avoiding any foaming, the contents of Mix Tank 1 is mixed until the Phospholipon 85G was uniformly dispersed. The polysorbate 80 is added to a suitable container (Mix Tank 2) and heated to between 5° and 60° C. The Ubidecarenone is then added to Mix Tank 2. While maintaining the temperature at between 5° and 60° C. Mix Tank 2 is mixed until all the Ubidecarenone is dissolved. After all the Ubidecarenone has been dissolved, the water phase is slowly transferred to Mix Tank 2. When all materials have been combined, the contents are homogenized until dispersion is smooth and uniform. While being careful not to overheat, the temperature is maintained at between 5° and 60° C. The homogenization is then stopped and the contents of Mix Tank 2 are transferred to a suitable container for storage.

In some embodiments, a formulation for any route of administration for use in the invention may include from about 0.001% to about 20% (w/w) of CoQ10, more preferably between about 0.01% and about 15% and even more preferably between about 0.1% to about 10% (w/w) of CoQ10. In certain embodiments, a formulation for any route of administration for use in the invention may include from about 1% to about 10% (w/w) of CoQ10. In certain embodiments, a formulation for any route of administration for use in the invention may include from about 2% to about 8% (w/w) of CoQ10. In certain embodiments, a formulation for any route of administration for use in the invention may include from about 2% to about 7% (w/w) of CoQ10. In certain embodiments, a formulation for any route of administration for use in the invention may include from about 3% to about 6% (w/w) of CoQ10. In certain embodiments, a formulation for any route of administration for use in the invention may include from about 3% to about 5% (w/w) of CoQ10. In certain embodiments, a formulation for any route of administration for use in the invention may include from about 3.5% to about 4.5% (w/w) of CoQ10. In certain embodiments, a formulation for any route of administration for use in the invention may include from about 3.5% to about 5% (w/w) of CoQ10. In one embodiment a formulation includes about 4% (w/w) of CoQ10. In one embodiment a formulation includes about 8% (w/w) of CoQ10. In one embodiment a formulation includes about 3% (w/w) of CoQ10. In various embodiments, the formulation includes about 0.1%, 0.2%, 0.3%, 0.4%, 0.5%, 1%, 2%, 3%, 4%, 5%, 6%, 7%, 8%, 9%, 10%, 11%, 12%, 13%, 14%, 15%, 16%, 17%, 18%, 19% or 20% (w/w) of CoQ10, or any range bracketed by any two values recited. In certain embodiments, the formulations can be prepared as a percent weight to volume rather than a percent weight to weight. Depending on the formulation, the concentration of CoQ10 may be the same, or about the same in the w/w and the w/v percent formulations. CoQ10 can be obtained from Kaneka Q10 as Kaneka Q10 (USP UBIDECARENONE) in powdered form (Pasadena, Texas, USA). CoQ10 used in the methods exemplified herein have the following characteristics: residual solvents meet USP 467 requirement; water content is less than 0.0%, less than 0.05% or less than 0.2%; residue on ignition is 0.0%, less than 0.05%, or less than 0.2%; heavy metal content is less than 0.002%, or less than 0.001%; purity of between 98-100% or 99.9%, or 99.5%.

In certain embodiments, the concentration of CoQ10 in the formulation is 1 mg/mL to 150 mg/mL. In one embodiment, the concentration of CoQ10 in the formulation is 5 mg/mL to 125 mg/mL. In one embodiment, the concentration of CoQ10 in the formulation is 10 mg/mL to 100 mg/mL. In one embodiment, the concentration of CoQ10 in the formulation is 20 mg/mL to 90 mg/mL. In one embodiment, the concentration of CoQ10 is 30 mg/mL to 80 mg/mL. In one embodiment, the concentration of CoQ10 is 30 mg/mL to 70 mg/mL. In one embodiment, the concentration of CoQ10 is 30 mg/mL to 60 mg/mL. In one embodiment, the concentration of CoQ10 is 30 mg/mL to 50 mg/mL. In one embodiment, the concentration of CoQ10 is 35 mg/mL to 45 mg/mL. It should be understood that additional ranges having any one of the foregoing values as the upper or lower limits are also intended to be part of this invention, e.g., 10 mg/mL to 50 mg/mL, or 20 mg/mL to 60 mg/mL.

In certain embodiments, the concentration of CoQ10 in the formulation is about 10, 15, 20, 25, 30, 31, 32, 33, 34, 35, 36, 37, 38, 39, 40, 41, 42, 43, 44, 45, 46, 47, 48, 49, 50, 55, 60, 65, 70, 75, 80, 85, 90 or 95 mg/mL. In one embodiment, the concentration of CoQ10 in the formulation is about 50 mg/mL. In one embodiment, the concentration of CoQ10 in the formulation is about 60 mg/mL. In one embodiment, the concentration of CoQ10 in the formulation is about 30 mg/mL. In a preferred embodiment, the concentration of CoQ10 in the formulation is about 40 mg/mL. It should be understood that ranges having any one of these values as the upper or lower limits are also intended to be part of this invention, e.g. between 37 mg/mL and 47 mg/mL, or between 31 mg/mL and 49 mg/mL.

It is understood that formulations can similarly be prepared containing CoQ10 precursors, metabolites, and related compounds.

### V. Methods of Treatment

In certain aspects, the disclosure relates to a method of treating a glioma in a subject comprising administering to the subject a composition comprising a Coenzyme Q10 compound, and radiation therapy, thereby treating the glioma in the subject.

In certain aspects, the disclosure relates to a method of treating a glioma in a subject comprising:
(a) administering a composition comprising Coenzyme Q10 to the subject by continuous intravenous infusion; and
(b) administering radiation therapy to the subject,
wherein the composition comprising Coenzyme Q10 is administered to the subject by continuous intravenous infusion for at least 24 hours before the radiation therapy is initiated, thereby treating the glioma in the subject.

### Administration of Coenzyme Q10

The Coenzyme Q10 may be administered to the subject before the radiation therapy is initiated to increase the efficacy of the radiation therapy. For example, in some embodiments, the composition comprising Coenzyme Q10 is administered to the subject by continuous intravenous infusion for at least 48 hours, at least 72 hours, at least 96 hours, at least 120 hours, or at least 144 hours before the radiation therapy is initiated. In some embodiments, the radiation therapy is initiated at least one week, at least two weeks, at least three weeks, at least four weeks, at least five weeks, at least six weeks, at least seven weeks, or at least eight weeks after administration of the composition comprising Coenzyme Q10 is initiated. In a particular embodiment, the radiation therapy is initiated at least one week after administration of the composition comprising Coenzyme Q10 is initiated. In a further particular embodiment, the radiation therapy is initiated at least two weeks after administration of the composition comprising Coenzyme Q10 is initiated.

In some embodiments, administration of the composition comprising Coenzyme Q10 is initiated 1 week before, 1-2 weeks before, 1-3 weeks before, 1-4 weeks before, 1-5 weeks before, or 1-6 weeks before the radiation therapy is initiated. In some embodiments, administration of the composition comprising Coenzyme Q10 is initiated 1-30 days, 1-25 days, 1-20 days, 1-15 days, 1-10 days, 1-9 days, 1-8 days, 1-7 days, 1-6 days, 1-5 days, 1-4 days, 1-3 days, or 1-2 days before the radiation therapy is initiated.

One or more doses of the composition comprising Coenzyme Q10 may be administered to the subject by continuous intravenous infusion before radiation therapy is initiated. For example at least 2, 3, 4, 5, 6, 7, 8, 9 or 10 doses of Coenzyme Q10 may be administered to the subject before radiation therapy is initiated. In a particular embodiment, one dose of the composition comprising Coenzyme Q10 is administered to the subject by continuous intravenous infusion before the radiation therapy is initiated. In a further particular embodiment, two doses of the composition comprising Coenzyme Q10 are administered to the subject by continuous intravenous infusion before the radiation therapy is initiated.

The doses of continuous intravenous infusion of Coenzyme Q10 may be administered at different intervals. For example, the doses of the composition comprising Coenzyme Q10 may be administered, for example, one time per week, two times per week, three times per week, four times per week, five times per week, six times per week, or seven times per week. In a particular embodiment, the composition comprising Coenzyme Q10 is administered once per week. In a further particular embodiment, the composition comprising Coenzyme Q10 is administered by continuous intravenous infusion (e.g. continuous intravenous infusion for about 96 hours) once per week.

In a further particular embodiment, two doses of the composition comprising Coenzyme Q10 are administered to the subject before radiation therapy is initiated, wherein the doses are administered once per week, and each dose comprises administering Coenzyme Q10 to the subject by continuous intravenous infusion for about 96 hours.

The composition comprising the Coenzyme Q10 compound (e.g. CoQ10) and the radiation therapy can act additively or synergistically. In some embodiments, the composition comprising the CoQ10 compound (e.g. CoQ10) and the radiation therapy are administered concurrently. In some embodiments, the composition comprising the CoQ10 compound (e.g. CoQ10) is administered prior to or subsequent to administration of the radiation therapy. In some embodiments, the CoQ10 compound (e.g. CoQ10) is administered prior to initiation of the radiation therapy, and administration of the CoQ10 compound (e.g. CoQ10) is then continued concurrently with the radiation therapy. In some embodiments, administration of the CoQ10 compound (e.g. CoQ10) is continued after radiation therapy is terminated.

In some embodiments, the composition comprising the Coenzyme Q10 compound (e.g. CoQ10) is administered by injection or infusion. In some embodiments, the composition comprising the Coenzyme Q10 compound (e.g. CoQ10) is administered intravenously. In a particular embodiment, the Coenzyme Q10 compound (e.g. Coenzyme Q10) is administered by continuous intravenous infusion. In other embodiments, the composition comprising the Coenzyme Q10 compound (e.g. CoQ10) is administered topically. In certain embodiments of the methods described herein, the subject is human. In some embodiments, the composition comprising the Coenzyme Q10 compound (e.g. CoQ10) is administered to the subject with an additional anti-cancer agent, for example, a chemotherapeutic agent or an anti-angiogenic agent.

In certain embodiments, formulations of the present disclosure may be utilized for the treatment of gliomas (e.g. glioblastomas) wherein the subject has failed treatment with at least one prior cancer therapeutic regimen, e.g., an anti-cancer agent, a chemotherapeutic regimen, or radiation therapy. In a particular embodiment, the at least one prior cancer therapeutic regimen comprises administration of temozolomide (TMZ) to the subject. In another particular embodiment, the at least one prior cancer therapeutic regimen comprises administration of bevacizumab to the subject. Accordingly, in some embodiments, the present invention provides a method of treating a glioma (e.g. glioblastoma) in a subject, wherein the subject has failed treatment for the glioma with radiation therapy, the method comprising administering to the subject a composition comprising a Coenzyme Q10 compound and radiation therapy, thereby treating the glioma in the subject. The methods of the invention may also be utilized for inhibiting glioma (e.g. glioblastoma) tumor cell growth in a subject wherein the subject has failed at least one prior cancer therapeutic regimen (e.g. a cancer therapeutic regimen comprising administration of TMZ and/or bevacizumab to the subject). Accordingly, the invention further provides methods of inhibiting glioma (e.g. glioblastoma) tumor cell growth in a subject, wherein the subject has failed at least one prior cancer therapeutic regimen, comprising administering a composition comprising a Coenzyme Q10 compound and radiation therapy to the subject, such that glioma tumor cell growth is inhibited. In a preferred embodiment, inhibiting glioma (e.g. glioblastoma) tumor growth includes achieving at least stable disease of the primary lesion by RECIST 1.1 criteria. In certain embodiments, the subject is a human subject.

In certain embodiments, formulations of the present disclosure may be utilized for the treatment of gliomas (e.g. glioblastomas) wherein the subject has not been previously treated with an anticancer agent and/or radiation therapy. In a particular embodiment, the anticancer agent is temozolomide (TMZ). In another particular embodiment, the anticancer agent is bevacizumab. Accordingly, in some embodiments, the present invention provides a method of treating a glioma (e.g. glioblastoma) in a subject, wherein the subject has not previously been treated for the glioma with an anticancer agent and/or radiation therapy, the method comprising administering to the subject a composition comprising a Coenzyme Q10 compound and radiation therapy, thereby treating the glioma in the subject. The methods of the invention may also be utilized for inhibiting glioma (e.g. glioblastoma) tumor cell growth in a subject wherein the subject has not previously been treated with an anticancer agent (e.g. TMZ and/or bevacizumab) for the glioma and/or radiation therapy for the glioma. Accordingly, the invention further provides methods of inhibiting glioma (e.g. glioblastoma) tumor cell growth in a subject, wherein the subject has not previously been treated with an anticancer agent (e.g. TMZ and/or bevacizumab) for the glioma and/or radiation therapy for the glioma, comprising administering a composition comprising a Coenzyme Q10 compound and radiation therapy to the subject, such that glioma tumor cell growth is inhibited. In a preferred embodiment, inhibiting glioma (e.g. glioblastoma) tumor growth includes achieving at least stable disease of the primary lesion by RECIST 1.1 criteria. In certain embodiments, the subject is a human subject.

The compositions described herein may include the hydrophobic therapeutic agent, e.g., CoQ10, its metabolites, or CoQ10 related compounds, in a pharmaceutically acceptable carrier. In some embodiments, such a composition may include from about 0.001% to about 20% (w/w) of CoQ10, between about 0.01% and about 15%, or between about 0.1% to about 10% (w/w) of CoQ10. In one embodiment a composition comprises about 4% (w/w) of CoQ10. In one embodiment a composition comprises about 8% (w/w) of CoQ10. In various embodiments, the composition comprises about 0.1%, 0.2%. 0.3%, 0.4%. 0.5%, 0.6%, 0.7%, 0.8%. 0.9%, 1%, 2%, 3%, 4%, 5%, 6%, 7%, 8%, 9%, 10%, 11%, 12%, 13%, 14%, 15%, 16%, 17%, 18%, 19% or 20% (w/w) of CoQ10, or any range bracketed by those values. In certain embodiments, the composition can be prepared as a percent weight to volume rather than a percent weight to weight. Depending on the formulation, the concentration of CoQ10 may be the same, or about the same in the w/w and the w/v percent formulations. As also noted herein, compositions of the present disclosure may be in a liquid form, capable of introduction into a subject by any means or route of administration within the purview of those skilled in the art. For example, compositions may be administered by routes of administration including, but not limited to, intravenous, intratumoral, combinations thereof, and the like.

In some embodiments, this disclosure relates to a method of treating glioma (e.g. glioblastoma or gliosarcoma) tumors in a human or other animal (e.g. a human or other animal that has failed at least one prior cancer therapeutic regimen) by administering to such human or animal an effective, non-toxic amount of a CoQ10 compound (e.g. CoQ10), for example, by administering an effective dose by IV administration, or, for example, by administering an effective dose by topical administration, in combination with radiation therapy. One skilled in the art would be able, by routine experimentation with the guidance provided herein, to determine what an effective, non-toxic amount of a CoQ10 compound would be for the purpose of treating a glioma in a subject who has failed at least one prior cancer therapeutic regimen. For example, a therapeutically active amount of the CoQ10 compound may vary according to factors such as the disease stage (e.g., stage I versus stage IV), age, sex, medical complications (e.g., immunosuppressed conditions or diseases) and weight of the subject, and the ability of the CoQ10 compound to elicit a desired response in the subject. The dosage regimen may be adjusted to provide the optimum therapeutic response. For example, several divided doses may be administered daily, the dose may be administered by continuous infusion, or the dose may be proportionally reduced as indicated by the exigencies of the therapeutic situation.

In certain embodiments of the invention, methods are provided for treating or preventing a glioma (e.g. glioblastoma) in a human (e.g. a human that was not previously treated with radiation therapy for the glioma and/or a chemotherapeutic agent for the glioma) by intravenously administering a composition comprising CoQ10, a CoQ10 precursor, metabolite, or related CoQ10 compound to the human such that treatment or prevention occurs, wherein the human is administered a dose of the composition such that, preferably, CoQ10 is administered in the range of about 0.5 mg/kg/dose to about 10,000 mg/kg/dose, about 5 mg/kg/dose to about 5,000 mg/kg/dose, about 10 mg/kg/dose to about 3,000 mg/kg/dose, about 10 mg/kg/dose to about 200 mg/kg/dose, or about 50 mg/kg/dose to about 150 mg/kg/dose. In one embodiment, the composition is administered such that CoQ10 is administered in the range of about 10 mg/kg/dose to about 1,400 mg/kg/dose. In one embodiment, the composition is administered such that, CoQ10 is administered in the range of about 10 mg/kg/dose to about 650 mg/kg/dose. In one embodiment, the composition is administered such that CoQ10 is administered in the range of about 50 mg/kg/dose to about 200 mg/kg/dose.

In certain embodiments of the invention, methods are provided for treating or preventing a glioma (e.g. glioblastoma) in a human (e.g. a human that has failed treatment for the glioma with radiation therapy, TMZ and/or bevacizumab) by intravenously administering a composition comprising CoQ10, a CoQ10 precursor, metabolite, or related CoQ10 compound to the human such that treatment or prevention occurs, wherein the human is administered a dose of the composition such that, preferably, CoQ10 is administered in the range of about 0.5 mg/kg/dose to about 10,000 mg/kg/dose, about 5 mg/kg/dose to about 5,000 mg/kg/dose, about 10 mg/kg/dose to about 3,000 mg/kg/dose, about 10 mg/kg/dose to about 200 mg/kg/dose, or about 50 mg/kg/dose to about 150 mg/kg/dose. In one embodiment, the composition is administered such that CoQ10 is administered in the range of about 10 mg/kg/dose to about 1,400 mg/kg/dose. In one embodiment, the composition is administered such that, CoQ10 is administered in the range of about 10 mg/kg/dose to about 650 mg/kg/dose. In one embodiment, the composition is administered such that CoQ10 is administered in the range of about 50 mg/kg/dose to about 200 mg/kg/dose.

In various embodiments, the composition is administered such that CoQ10 is administered at a dose of about 2 mg/kg/dose, 5 mg/kg/dose, 10 mg/kg/dose, 15 mg/kg/dose, 20 mg/kg/dose, 25 mg/kg/dose, 30 mg/kg/dose, 35 mg/kg/dose, 40 mg/kg/dose, 45 mg/kg/dose, 50 mg/kg/dose, 55 mg/kg/dose, 56 mg/kg/dose, 57 mg/kg/dose, 58 mg/kg/dose, 59 mg/kg/dose, 60 mg/kg/dose, 65 mg/kg/dose, 70 mg/kg/dose, 75 mg/kg/dose, 76 mg/kg/dose, 77 mg/kg/dose, 78 mg/kg/dose, 79 mg/kg/dose, 80 mg/kg/dose, 85 mg/kg/dose, 88 mg/kg/dose, 90 mg/kg/dose, 95 mg/kg/dose, 100 mg/kg/dose, 101 mg/kg/dose, 102 mg/kg/dose, 103 mg/kg/dose, 104 mg/kg/dose, 105 mg/kg/dose, 106 mg/kg/dose, 107 mg/kg/dose, 108 mg/kg/dose, 109 mg/kg/dose, 110 mg/kg/dose, 120 mg/kg/dose, 130 mg/kg/dose, 140 mg/kg/dose, 150 mg/kg/dose, 160 mg/kg/dose, 170 mg/kg/dose, 180 mg/kg/dose, 190 mg/kg/dose, 200 mg/kg/dose, 210 mg/kg/dose, 220 mg/kg/dose, 250 mg/kg/dose, 275 mg/kg/dose, 300 mg/kg/dose, 325 mg/kg/dose, 350 mg/kg/dose, 375 mg/kg/dose, 400 mg/kg/dose, 450 mg/kg/dose, 500 mg/kg/dose, 550 mg/kg/dose, 600 mg/kg/dose or 675 mg/kg/dose. In certain embodiments, the Coenzyme Q10 is administered at a dose selected from the group consisting of about 50 mg/kg/dose, about 66 mg/kg/dose, about 88 mg/kg/dose, about 110 mg/kg/dose, about mg/kg/dose, about 171 mg/kg/dose, about 215 mg/kg/dose, about 286 mg/kg/dose, about 380 mg/kg/dose, about 505 mg/kg/dose, and about 672 mg/kg/dose. In a particular embodiment, the Coenzyme Q10 is administered at a dose of about 88 mg/kg/dose. In a particular embodiment, the Coenzyme Q10 is administered at a dose of about 110 mg/kg/dose. In various embodiments, the dose is administered by continuous infusion for at least 48 hours, at least 72 hours or at least 96 hours. In various embodiments, the dose is administered by continuous infusion for about 48 hours, about 72 hours or about 96 hours.

In various embodiments, the composition is administered such that CoQ10 is administered at a dose of at least 2 mg/kg/dose, 5 mg/kg/dose, 10 mg/kg/dose, 15 mg/kg/dose, 20 mg/kg/dose, 25 mg/kg/dose, 30 mg/kg/dose, 35 mg/kg/dose, 40 mg/kg/dose, 45 mg/kg/dose, 50 mg/kg/dose, 55 mg/kg/dose, 56 mg/kg/dose, 57 mg/kg/dose, 58 mg/kg/dose, 59 mg/kg/dose, 60 mg/kg/dose, 65 mg/kg/dose, 70 mg/kg/dose, 75 mg/kg/dose, 76 mg/kg/dose, 77 mg/kg/dose, 78 mg/kg/dose, 79 mg/kg/dose, 80 mg/kg/dose, 85 mg/kg/dose, 88 mg/kg/dose, 90 mg/kg/dose, 95 mg/kg/dose, 100 mg/kg/dose, 101 mg/kg/dose, 102 mg/kg/dose, 103 mg/kg/dose, 104 mg/kg/dose, 105 mg/kg/dose, 106 mg/kg/dose, 107 mg/kg/dose, 108 mg/kg/dose, 109 mg/kg/dose, 110 mg/kg/dose, 120 mg/kg/dose, 130 mg/kg/dose, 140 mg/kg/dose, 150 mg/kg/dose, 160 mg/kg/dose, 170 mg/kg/dose, 180 mg/kg/dose, 190 mg/kg/dose, or 200 mg/kg/dose, 210 mg/kg/dose, 220 mg/kg/dose, 250 mg/kg/dose, 275 mg/kg/dose, 300 mg/kg/dose, 325 mg/kg/dose, 350 mg/kg/dose, 375 mg/kg/dose, 400 mg/kg/dose, 450 mg/kg/dose, 500 mg/kg/dose, 550 mg/kg/dose, 600 mg/kg/dose or 675 mg/kg/dose, wherein the dose does not result in any limiting toxicities. It should be understood that ranges having any one of these values as the upper or lower limits are also intended to be part of this invention, e.g., about 50 mg/kg/dose to about 200 mg/kg/dose, or about 650 mg/kg/dose to about 1400 mg/kg/dose, or about 55 mg/kg/dose to about 110 mg/kg/dose. In a particular embodiment, the Coenzyme Q10 is administered at a dose of at least 88 mg/kg/dose. In a particular embodiment, the Coenzyme Q10 is administered at a dose of at least 110 mg/kg/dose. In various embodiments, the dose is administered by continuous infusion for at least 48 hours, at least 72 hours or at least 96 hours. In various embodiments, the dose is administered by continuous infusion for about 48 hours, about 72 hours or about 96 hours.

In one embodiment the administered dose of Coenzyme Q10 is at least about 1 mg/kg/dose, at least about 5 mg/kg/dose, at least about 10 mg/kg/dose, at least about 12.5 mg/kg/dose, at least about 20 mg/kg/dose, at least about 25 mg/kg/dose, at least about 30 mg/kg/dose, at least about 35 mg/kg/dose, at least about 40 mg/kg/dose, at least about 45 mg/kg/dose, at least about 50 mg/kg/dose, at least about 55 mg/kg/dose, at least about 60 mg/kg/dose, at least about 75 mg/kg/dose, at least about 100 mg/kg/dose, at least about 125 mg/kg/dose, at least about 150 mg/kg/dose, at least about 175 mg/kg/dose, at least about 200 mg/kg/dose, at least about 250 mg/kg/dose, at least about 300 mg/kg/dose, at least about 350 mg/kg/dose, at least about 400 mg/kg/dose, at least about 450 mg/kg/dose, at least about 500 mg/kg/dose, at least about 550 mg/kg/dose, at least about 600 mg/kg/dose, or at least about 600 mg/kg/dose. In certain embodiments, the Coenzyme Q10 is administered at a dose selected from the group consisting of at least 50 mg/kg/dose, at least 66 mg/kg/dose, at least 88 mg/kg/dose, at least 110 mg/kg/dose, at least 137 mg/kg/dose, at least 171 mg/kg/dose, at least 215 mg/kg/dose, at least 286 mg/kg/dose, at least 380 mg/kg/dose, at least 505 mg/kg/dose, or at least 672 mg/kg/dose. In various embodiments, the dose is administered by continuous infusion over at least 48 hours, at least 72 hours or at least 96 hours. In various embodiments, the dose is administered by continuous infusion over about 48 hours, about 72 hours or about 96 hours.

In certain embodiments, the administered dose is no more than about 20 mg/kg/dose, about 25 mg/kg/dose, about 30 mg/kg/dose, about 35 mg/kg/dose, about 40 mg/kg/dose, about 45 mg/kg/dose, about 50 mg/kg/dose, about 55 mg/kg/dose, about 60 mg/kg/dose, about 75 mg/kg/dose, about 100 mg/kg/dose, about 125 mg/kg/dose, about 150 mg/kg/dose, about 175 mg/kg/dose, about 200 mg/kg/dose, about 300 mg/kg/dose, about 400 mg/kg/dose, about 500 mg/kg/dose, about 600 mg/kg/dose, about 700 mg/kg/dose, about 800 mg/kg/dose, about 900 mg/kg/dose, about 1000 mg/kg/dose, about 1100 mg/kg/dose, about 1200 mg/kg/dose, or about 1300 mg/kg/dose. In various embodiments, the dose is administered by continuous infusion over at least 48 hours, at least 72 hours or at least 96 hours. In various embodiments, the dose is administered by continuous infusion over about 48 hours, about 72 hours or about 96 hours.

In all of the foregoing aspects, it is understood that any of the lower limit values and upper limit values can be combined to create a range. In certain embodiments, the administered dose is at least 75 mg/kg/dose or 100 mg/kg/dose or the rat equivalent to about, at least, 12.2 or 16.2 mg/kg/day in humans, or at least 85 mg/kg over a week period, or at least 113 mg/kg over a week period.

In certain embodiments, the Coenzyme Q10 is administered at a dose selected from the group consisting of about 11.8 mg/kg/day (24 hours), about 12.5 mg/kg/day (24 hours), about 14.4 mg/kg/day (24 hours), about 15.6 mg/kg (24 hours), about 16.5 mg/kg/day (24 hours), about 19 mg/kg/day (24 hours), about 20.4 mg/kg/day (24 hours), about 22 mg/kg/day (24 hours), about 25 mg/kg/day (24 hours), about 27.5 mg/kg/day (24 hours), about 29.3 mg/kg/day (24 hours), about 33 mg/kg/day (24 hours), about 34.2 mg/kg/day (24 hours), about 36.7 mg/kg/day (24 hours), about 41.7 mg/kg/day (24 hours), 42.8 mg/kg/day (24 hours), about 44 mg/kg/day (24 hours), about 45.7 mg/kg/day (24 hours), about 51.9 mg/kg/day (24 hours), about 53.8 mg/kg/day (24 hours), about 55 mg/kg/day (24 hours), about 57 mg/kg/day (24 hours), about 58.7 mg/kg/day (24 hours), about 64.8 mg/kg/day (24 hours), about 66.7 mg/kg/day (24 hours), about 68.5 mg/kg/day (24 hours), about 71.7 mg/kg/day (24 hours), about 73.4 mg/kg/day (24 hours), about 81.5 mg/kg/day (24 hours), about 85.5 mg/kg/day (24 hours), about 91.7 mg/kg/day (24 hours), about 107.5 mg/kg/day (24 hours), about 114.6 mg/kg/day (24 hours), and about 143.3 mg/kg/day (24 hours).

In certain embodiments, the Coenzyme Q10 is administered at a dose selected from the group consisting of at least 15.5 mg/kg/day (24 hours), at least 16.7 mg/kg/day (24 hours), at least 19.0 mg/kg/day (24 hours), at least 20.5 mg/kg/day (24 hours), at least 22.0 mg/kg/day (24 hours), at least 25.0 mg/kg/day (24 hours), at least 27.3 mg/kg/day (24 hours), at least 29.3 mg/kg/day (24 hours), at least 33.4 mg/kg/day (24 hours), at least 36.7 mg/kg/day (24 hours), at least 34.1 mg/kg/day (24 hours), at least 41.7 mg/kg/day (24 hours), at least 42.5 mg/kg/day (24 hours), at least 45.7 mg/kg/day (24 hours), at least 52.0 mg/kg/day (24 hours), at least 53.1 mg/kg/day (24 hours), at least 57 mg/kg/day (24 hours), at least 64.9 mg/kg/day (24 hours), at least 66.7 mg/kg/day (24 hours), at least 71.7 mg/kg/day (24 hours), at least 81.5 mg/kg/day (24 hours), at about 88.8 mg/kg/day (24 hours), at least 95.3 mg/kg/day (24 hours), at least 108.5 mg/kg/day (24 hours), at least 117.9 mg/kg/day (24 hours), at least 126.7 mg/kg/day (24 hours), at least 144.2 mg/kg/day (24 hours), at least 156.7 mg/kg/day (24 hours), at least 168.3 mg/kg/day (24 hours), at least 191.6 mg/kg/day (24 hours), at least 208.5 mg/kg/day (24 hours), at least 224 mg/kg/day (24 hours), and at least 254.9 mg/kg/day (24 hours).

In certain embodiments, the Coenzyme Q10 is administered at a dose selected from the group consisting of about 15.5 mg/kg/day (24 hours), about 16.7 mg/kg/day (24 hours), about 19.0 mg/kg/day (24 hours), about 20.5 mg/kg/day (24 hours), about 22.0 mg/kg/day (24 hours), about 25.0 mg/kg/day (24 hours), about 27.3 mg/kg/day (24 hours), about 29.3 mg/kg/day (24 hours), about 33.4 mg/kg/day (24 hours), about 36.7 mg/kg/day (24 hours), about 34.1 mg/kg/day (24 hours), about 41.7 mg/kg/day (24 hours), about 42.5 mg/kg/day (24 hours), about 45.7 mg/kg/day (24 hours), about 52.0 mg/kg/day (24 hours), about 53.1 mg/kg/day (24 hours), about 57 mg/kg/day (24 hours), about 64.9 mg/kg/day (24 hours), about 66.7 mg/kg/day (24 hours), about 71.7 mg/kg/day (24 hours), about 81.5 mg/kg/day (24 hours), about 88.8 mg/kg/day (24 hours), about 95.3 mg/kg/day (24 hours), about 108.5 mg/kg/day (24 hours), about 117.9 mg/kg/day (24 hours), about 126.7 mg/kg/day (24 hours), about 144.2 mg/kg/day (24 hours), about 156.7 mg/kg/day (24 hours), about 168.3 mg/kg/day (24 hours), about 191.6 mg/kg/day (24 hours), about 208.5 mg/kg/day (24 hours), about 224 mg/kg/day (24 hours), and about 254.9 mg/kg/day (24 hours).

In certain embodiments, the Coenzyme Q10 is administered at a dose selected from the group consisting of about 38 mg/kg/week, about 50 mg/kg/week, about 66 mg/kg/week, about 76 mg/kg/week, about 88 mg/kg/week, about 100 mg/kg/week, about 110 mg/kg/week, about 132 mg/kg/week, about 137 mg/kg/week, about 171 mg/kg/week, about 176 mg/kg/week, about 215 mg/kg/week, about 220 mg/kg/week, about 274 mg/kg/week, about 342 mg/kg week, and about 430 mg/kg/week. In certain embodiments, the Coenzyme Q10 is administered at a dose selected from the group consisting of about 100 mg/kg/week, about 132 mg/kg/week, about 171 mg/kg/week, about 215 mg/kg/week, about 274 mg/kg/week, about 430 mg/kg/week, about 572 mg/kg/week, about 760 mg/kg/week, about 1010 mg/kg/week, and about 1344 mg/kg/week.

In certain embodiments, the Coenzyme Q10 is administered at a dose selected from the group consisting of at least 88 mg/kg/week, at least 100 mg/kg/week, at least 110 mg/kg/week, at least 132 mg/kg/week, at least 171 mg/kg/week, at least 215 mg/kg/week, at least 274 mg/kg/week, at least 430 mg/kg/week, at least 572 mg/kg/week, at least 760 mg/kg/week, at least 1010 mg/kg/week, and at least 1344 mg/kg/week.

In a particular embodiment, two doses of the composition comprising Coenzyme Q10 are administered to the subject before radiation therapy is initiated, wherein the doses are administered once per week, and each dose comprises administering Coenzyme Q10 to the subject by continuous intravenous infusion for about 96 hours in an amount of about 110 mg/kg/week.

In some embodiments, the weekly dose of Coenzyme Q10 is administered by two consecutive continuous infusions for about 72 hours each.

In one embodiment, the CoQ10 composition is administered one time per week, for example, one time per week for at least 24, 48, 72, 96, or 120 hours. In a particular embodiment, the CoQ10 composition is administered to the subject by continuous intravenous infusion once per week for at least 96 hours. In one embodiment, the CoQ10 composition is administered 2 times per week. In one embodiment, the CoQ10 composition is administered 3 times per week. In one embodiment, the CoQ10 composition is administered 4 times per week. In one embodiment, the CoQ10 composition is administered 5 times per week. In one embodiment, the CoQ10 composition is administered 6 times per week. In one embodiment, the CoQ10 composition is administered once per day. In some embodiments, where the CoQ10 composition is an IV formulation administered by infusion, the dosage is administered by infusion for about 1 hour, for about 2 hours, for about 3 hours, for about 4 hours, or longer. In one embodiment, the CoQ10 composition is administered by infusion for about 4 hours, e.g., about 3.5 hours to about 4.5 hours. In certain embodiments, the formulation is administered for 4 or more hours. In certain embodiments, the formulation is administered for 8 or more hours. In certain embodiments, the formulation is administered for 12 hours or more. In certain embodiments, the formulation is administered for 18 or more hours. In certain embodiments, the formulation is administered for 24 or more hours. In certain embodiments, the formulation is administered for about 24 hours.

In some embodiments, the continuous intravenous infusion of Coenzyme Q10 is administered for at least 48 hours, at least 72 hours, at least 96 hours, at least 120 hours, or at least 144 hours or more. In some embodiments, the continuous intravenous infusion of Coenzyme Q10 is administered for about 48 hours, about 72 hours, about 96 hours, about 120 hours, or about 144 hours. In a particular embodiment, the continuous intravenous infusion of Coenzyme Q10 is administered for at least 96 hours. In a particular embodiment, the continuous intravenous infusion of Coenzyme Q10 is administered for about 96 hours.

In certain embodiments, the formulation, preferably, a CoQ10 formulation, can be administered in one or more cycles. For example, the CoQ10 can be administered for 2, 3, 4, 5, 6, 7, 8, or more weeks consecutively, and then not administered for a period of 1, 2, 3, 4, or more weeks, providing a cycle of administration. In certain embodiments, the cycles are administered without a pause between cycles. In certain embodiments, at the end of one or more cycles, the patient is assessed to determine treatment efficacy, toxicity, and assess if the treatment should be continued, modified, or ended. The number of cycles of administration depends, for example, on the response of the subject, the severity of disease, other therapeutic interventions used on the subject, or any adverse response of the subject. In certain embodiments, the CoQ10 formulation is administered as long as the subject is exhibiting at least a stable response to treatment with no serious adverse events, e.g., dose limiting toxicities, grade IV toxicities, or persistent grade III toxicities that cannot be mitigated by the use of other interventions.

In another embodiment, the formulation, preferably, a CoQ10 formulation, is administered in the form of a CoQ10 IV formulation at a dosage of between about 10 mg/kg/dose and about 10,000 mg/kg/dose of CoQ10, about 20 mg/kg/dose to about 5000 mg/kg/dose, about 50 mg/kg/dose to about 3000 mg/kg/dose, about 100 mg/kg/dose to about 2000 mg/kg/dose, about 200 mg/kg/dose to about 1000 mg/kg/dose, about 300 mg/kg/dose to about 500 mg/kg/dose, or about 55 mg/kg/dose to about 110 mg/kg/dose wherein the CoQ10 formulation comprises between about 1% and 10% of CoQ10 (w/v). In one embodiment, the CoQ10 formulation comprises about 4% of CoQ10 (w/v). In one embodiment, the CoQ10 IV formulation comprises about 8% of CoQ10 (w/v). In other embodiments, the CoQ10 IV formulation comprises about 0.1%, 0.2%. 0.3%, 0.4%. 0.5%, 0.6%, 0.7%, 0.8%. 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5% or 10% of CoQ10 (w/v). It should be understood that ranges having any one of these values as the upper or lower limits are also intended to be part of this invention.

In the treatment of a glioma (e.g. a glioblastoma) according to the methods of the invention, the compositions may be in a pharmaceutically acceptable carrier that may be administered in a therapeutically effective amount to a subject in combination with radiation therapy and at least one other anticancer agent, e.g., chemotherapeutic agent, for a given indication, following surgical intervention to radically remove a tumor, in combination with other alternative and/or complementary acceptable treatments for cancer, and the like.

In certain embodiments, the effect a CoQ10 compound may have on glioma (e.g. glioblastoma or gliosarcoma) cells, according to the methods of the invention may depend, in part, on the various states of metabolic and oxidative flux exhibited by the glioma cells. A CoQ10 compound of the invention may be utilized to interrupt and/or interfere with the conversion of an oncogenic cell's dependency of glycolysis and increased lactate utility. As it relates to a cancer state, this interference with the glycolytic and oxidative flux of the tumor microenvironment may influence apoptosis and angiogenesis in a manner which reduces the viability or proliferative capacity of a cancer cell. In some embodiments, the interaction of a CoQ10 compound with glycolytic and oxidative flux factors may enhance the ability of the CoQ10 compound to exert its restorative apoptotic effect in the particular cancer, e.g., glioma (e.g. glioblastoma or gliosarcoma).

While the present disclosure has focused on CoQ10 and its metabolites, other compounds related to CoQ10 which may be administered instead of, or in combination with, CoQ10 include, but are not limited to, benzoquinones, isoprenoids, farnesols, farnesyl acetate, farnesyl pyrophosphate, 1-phenylalanine, d-phenylalanine, dl-phenylalanine, 1-tyrosine, d-tyrosine, dl-tyrosine, 4-hydroxy-phenylpyruvate, 4-hydroxy-phenyllactate, 4-hydroxy-cinnamate, dipeptides and tripeptides of tyrosine or phenylalanine, 3,4-dihydroxymandelate, 3-methoxy-4-hydroxyphenylglycol, 3-methoxy-4-hydroxymandelate, vanillic acid, phenylacetate, pyridoxine, S-adenosyl methionine, panthenol, mevalonic acid, isopentyl pyrophosphate, phenylbutyrate, 4-hydroxy-benzoate, decaprenyl pyrophosphate, beta-hydroxybutyrate, 3-hydroxy-3-methyl-glutarate, acetylcarnitine, acetoacetylcarnitine, acetylglycine, acetoacetylglycine, carnitine, acetic acid, pyruvic acid, 3-hydroxy-3-methylglutarylcarnitine, all isomeric forms of serine, alanine, cysteine, glycine, threonine, hydroxyproline, lysine, isoleucine, and leucine, even carbon number C₄ to C₈ fatty acids (butyric, caproic, caprylic, capric, lauric, myristic, palmitic, and stearic acids) salts of carnitine and glycine, e.g., palmitoylcarnitine and palmitoylglycine, and 4-hydroxy-benzoate polyprenyltransferase, any salts of these compounds, as well as any combinations thereof, and the like. It is understood that such treatment methods can similarly be performed by administration of other CoQ10 precursors, metabolites, and related compounds.

### Radiation Therapy

The composition comprising Coenzyme Q10 may be administered to the subject in combination with radiation therapy. Radiation therapies for glioma are known in the art and are described, for example, in Cabrera et al., 2016, Practical Radiation Oncology 6: 217-225, which is incorporated by reference herein in its entirety. Techniques for delivering radiation therapy to gliomas include, but are not limited to, external beam radiation therapy (EBRT), stereotactic radiation therapy (SRT), and internal radiation therapy (brachytherapy).

EBRT involves directing radiation beams from outside the body into the tumor. Linear accelerators produce the high-energy radiation beams that penetrate the tissues and deliver the radiation dose directly to the cancer. EBRT is typically delivered as an outpatient procedure for approximately six to eight weeks. In some embodiments, the EBRT is three-dimensional conformal radiation therapy (3D-CRT). 3D-CRT can be delivered more precisely with the help of a computed tomography (CT scan) and a computer. The CT scan is used to identify the glioma tumor, and the computer is used to “conform” the radiation to the glioma tumor shape. The use of 3D-CRT appears to reduce the chance of injury to nearby normal tissues. EBRT may be used to deliver radiation therapy to a part of the brain or the whole-brain. Whole-brain radiation therapy (WBRT) is usually recommended for a large or spreading brain tumor. In some embodiments, the EBRT is proton radiation therapy. Proton radiation therapy is a form of EBRT that utilizes a beam of protons as the source of radiation rather than X-rays or gamma rays. Protons are released from atoms using technology similar to that employed in nuclear reactors. Computer-programmed blocks are precisely placed to direct the proton beam toward the tumor and match it to the shape of the tumor.

Stereotactic radiation therapy (SRT) is a noninvasive approach to the treatment of brain tumors that uses pencil-thin beams of radiation to treat only the tumor. SRT uses imaging techniques—including CT scans or MRI—and special computerized planning to precisely focus a high dose of radiation on the brain tumor, while sparing normal tissue. This focused technique allows radiation to be delivered in an area of the brain or spinal cord that might be considered inoperable, and can be delivered to tumors that are one and one-half inches in diameter or smaller. Another major advantage to SRT is that radiation treatment is delivered in a single session.

Internal Radiation Therapy (Brachytherapy) is the delivery of radiation therapy by placing radioactive material directly into or near the brain tumor. The radioactive material may also be called “implants” or “seeds.” While standard radiation aims rays at the tumor from outside the body, brachytherapy attacks the tumor from the inside. Brachytherapy is used in the treatment of newly diagnosed or recurrent brain tumors. It may be administered as the primary radiation therapy or as a “boost” of additional radiation delivered before or following standard external beam radiation. Brachytherapy is a local therapy; it is not commonly used for widely spread or multiple tumors. To implant radiation energy in the tumor, catheters are placed into the tumor bed using surgical techniques that are directed by CT and MRI. The sources of radiation, usually in pellet form, are placed in the catheters. Depending on the isotopes used, the implant is removed either after a few days, after several months or is left in place permanently. Steroids are commonly used with this therapy to decrease brain swelling. In some embodiments, implantation may be repeated.

In some embodiments, the radiation therapy is administered to the subject for at least 2, 3, 4, 5, 6, 7 or 8 weeks. In a particular embodiment, the radiation therapy is administered to the subject for at least 6 weeks. In some embodiments, at least 5, 10, 20, 30, 40, 50 or 60 doses of radiation are administered to the subject. In a particular embodiment, at least 30 doses of radiation are administered to the subject.

The dose of radiation administered to the subject may be measured as a daily dose or as a total dose. The “total dose” as described herein refers to the total amount of radiation administered to a subject over several days or weeks. In some embodiments the daily dose of radiation administered to the subject is about 0.1, 0.2, 0.3, 0.4, 0.5, 1, 1.1, 1.2, 1.3, 1.4, 1.5, 1.6, 1.7, 1.8, 1.9, 2, 2.1, 2.2, 2.3, 2.4, 2.5, 3, 4, 5, 6, 7, 8, 9, 10, 11, 12, 13, 14, 15, 16, 17, 18, 19 or 20 Gy. In some embodiments the daily dose of radiation administered to the subject is at least 0.1, 0.2, 0.3, 0.4, 0.5, 1, 1.1, 1.2, 1.3, 1.4, 1.5, 1.6, 1.7, 1.8, 1.9, 2, 2.1, 2.2, 2.3, 2.4, 2.5, 3, 4, 5, 6, 7, 8, 9, 10, 11, 12, 13, 14, 15, 16, 17, 18, 19 or 20 Gy. In some embodiments, the daily dose of radiation administered to the subject is less than 0.1, 0.2, 0.3, 0.4, 0.5, 1, 1.1, 1.2, 1.3, 1.4, 1.5, 1.6, 1.7, 1.8, 1.9, 2, 2.1, 2.2, 2.3, 2.4, 2.5, 3, 4, 5, 6, 7, 8, 9, 10, 11, 12, 13, 14, 15, 16, 17, 18, 19 or 20 Gy. Any of these values may be used to define a range for the daily dose of radiation administered to the subject. For example, in some embodiments, the daily dose of radiation administered to the subject is 0.1 to 20 Gy, 1 to 10 Gy, 5 to 15 Gy, 21 to 100 Gy, or 0.5 to 3 Gy. In a particular embodiment, the daily dose of radiation administered to the subject is about 1.8 to 2 Gy.

In some embodiments, the total dose of radiation administered to the subject is about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 70, 80, 90, 100, 150 or 200 Gy. In some embodiments, the total dose of radiation administered to the subject is at least 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 70, 80, 90, 100, 150 or 200 Gy. In some embodiments, the total dose of radiation administered to the subject is less than 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 70, 80, 90, 100, 150 or 200 Gy. Any of these values may be used to define a range for the dose of radiation administered to the subject. For example, in some embodiments, the total dose of radiation administered to the subject is 0.1 to 20 Gy, 1 to 10 Gy, 5 to 15 Gy, 21 to 70 Gy, or 21 to 100 Gy. In a particular embodiment, the total dose of radiation administered to the subject is at least 21 Gy. In a further particular embodiment, the total dose of radiation administered to the subject is about 60 Gy.

The radiation therapy may be administered to the subject at a dose rate of 10, 20, 30, 40, 50, 60, 70, 80, 90, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 4000 or 5000 cGy/min. In some embodiments, the radiation therapy dose rate is at least 10, 20, 30, 40, 50, 60, 70, 80, 90, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 4000 or 5000 cGy/min. In some embodiments, the radiation therapy dose rate is less than 10, 20, 30, 40, 50, 60, 70, 80, 90, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 4000 or 5000 cGy/min. Any of these values may be used to define a range for the radiation therapy dose rate. For example, in some embodiments, the radiation therapy dose rate is 10-100 cGy/min, 100-2400 cGy/min, or 500-2400 cGy/min.

In some embodiments, an amount of irradiation at a total dose as measured over one or a plurality of radiation treatments in a 24 hour period does not exceed 1 Gy at a dose rate of 2,400 cGy/min.

### Additional Cancer Therapies

In certain embodiments, the compositions comprising a CoQ10 compound (e.g. CoQ10) and radiation therapies disclosed herein can be used in further combination with at least one additional cancer therapy.

In some embodiments, the additional cancer therapy comprises or consists of a wave-like electric field call a Tumor Treating Field administered with an Optune® device. Optune® is a wearable, portable, FDA-approved treatment for glioblastoma multiforme (GBM) that creates low-intensity, wave-like electric fields called Tumor Treating Fields (TTFields), which interfere with GBM cell division. The Optune® device attaches to a patient's scalp via four adhesive transducer arrays which are about palm-sized and are placed adjacent to one another. The arrays develop an alternating electrical field between opposing arrays that slows or stops GBM tumor cells from dividing and may destroy them. Specifically, the electrical field disorganizes the way the cell microtubules are constructed, and prevents the GBM tumor cell from dividing into daughter cells.

In some embodiments, the additional cancer therapy comprises or consists of one or more anticancer agents. The anticancer agents may include, e.g., chemotherapeutic agents, (e.g., small molecule anticancer agents), or biologic anticancer agents including both protein based and nucleic acid based therapeutics. In one embodiment, an additional anticancer agent for use in the therapeutic methods of the invention is a chemotherapeutic agent, e.g., TMZ. In some embodiments, the TMZ is administered at a dose of about 75 mg/m² once per day. In some embodiments the TMZ is administered for at least 5, 10, 15, 20, 25, 30, 35 or 40 days. In a particular embodiment, the TMZ is administered for about 42 days. In one embodiment, an additional anticancer agent for use in the therapeutic methods of the invention is an antibody, e.g., bevacizumab. In some embodiments, the CoQ10 compound (e.g. CoQ10) is administered in an amount that would be therapeutically effective to treat glioma, i.e., the CoQ10 compound (e.g. CoQ10) is administered and/or acts as a therapeutic agent for the glioma, and not predominantly as an agent to ameliorate side effects of other chemotherapy or other cancer treatments. The CoQ10 compound (e.g. CoQ10), radiation therapy, and the additional therapeutic (anticancer) agent can act additively or synergistically.

Chemotherapeutic agents generally belong to various classes including, for example: (1) Topoisomerase II inhibitors (cytotoxic antibiotics), such as the anthracyclines/anthracenediones, e.g., doxorubicin, epirubicin, idarubicin and nemorubicin, the anthraquinones, e.g., mitoxantrone and losoxantrone, and the podophillotoxines, e.g., etoposide and teniposide; (2) Agents that affect microtubule formation (mitotic inhibitors), such as plant alkaloids (e.g., a compound belonging to a family of alkaline, nitrogen-containing molecules derived from plants that are biologically active and cytotoxic), e.g., taxanes, e.g., paclitaxel and docetaxel, and the vinka alkaloids, e.g., vinblastine, vincristine, and vinorelbine, and derivatives of podophyllotoxin; (3) Alkylating agents, such as nitrogen mustards, ethyleneimine compounds, alkyl sulphonates and other compounds with an alkylating action such as nitrosoureas, dacarbazine, cyclophosphamide, ifosfamide and melphalan; (4) Antimetabolites (nucleoside inhibitors), for example, folates, e.g., folic acid, fiuropyrimidines, purine or pyrimidine analogues such as 5-fluorouracil, capecitabine, gemcitabine, methotrexate, and edatrexate; (5) Topoisomerase I inhibitors, such as topotecan, irinotecan, and 9-nitrocamptothecin, camptothecin derivatives, and retinoic acid; and (6) Platinum compounds/complexes, such as cisplatin, oxaliplatin, and carboplatin.

Exemplary chemotherapeutic agents for use in the methods of the invention include, but are not limited to, amifostine (ethyol), cisplatin, dacarbazine (DTIC), dactinomycin, mechlorethamine (nitrogen mustard), streptozocin, cyclophosphamide, carrnustine (BCNU), lomustine (CCNU), doxorubicin (adriamycin), doxorubicin lipo (doxil), gemcitabine (gemzar), daunorubicin, daunorubicin lipo (daunoxome), procarbazine, mitomycin, cytarabine, etoposide, methotrexate, 5-fluorouracil (5-FU), vinblastine, vincristine, bleomycin, paclitaxel (taxol), docetaxel (taxotere), aldesleukin, asparaginase, busulfan, carboplatin, cladribine, camptothecin, CPT-I 1, lO-hydroxy-7-ethyl-camptothecin (SN38), dacarbazine, S-I capecitabine, ftorafur, 5′deoxyflurouridine, UFT, eniluracil, deoxycytidine, 5-azacytosine, 5-azadeoxycytosine, allopurinol, 2-chloro adenosine, trimetrexate, aminopterin, methylene-10-deazaaminopterin (MDAM), oxaplatin, picoplatin, tetraplatin, satraplatin, platinum-DACH, ormaplatin, CI-973, JM-216, and analogs thereof, epirubicin, etoposide phosphate, 9-aminocamptothecin, 10, 11-methylenedioxycamptothecin, karenitecin, 9-nitrocamptothecin, TAS 103, vindesine, L-phenylalanine mustard, ifosphamidemefosphamide, perfosfamide, trophosphamide carmustine, semustine, epothilones A-E, tomudex, 6-mercaptopurine, 6-thioguanine, amsacrine, etoposide phosphate, karenitecin, acyclovir, valacyclovir, ganciclovir, amantadine, rimantadine, lamivudine, zidovudine, bevacizumab, trastuzumab, rituximab, 5-Fluorouracil, Capecitabine, Pentostatin, Trimetrexate, Cladribine, floxuridine, fludarabine, hydroxyurea, ifosfamide, idarubicin, mesna, irinotecan, mitoxantrone, topotecan, leuprolide, megestrol, melphalan, mercaptopurine, plicamycin, mitotane, pegaspargase, pentostatin, pipobroman, plicamycin, streptozocin, tamoxifen, teniposide, testolactone, thioguanine, thiotepa, uracil mustard, vinorelbine, chlorambucil, cisplatin, doxorubicin, paclitaxel (taxol), bleomycin, mTor, epidermal growth factor receptor (EGFR), and fibroblast growth factors (FGF) and combinations thereof which are readily apparent to one of skill in the art based on the appropriate standard of care for a particular tumor or cancer.

In another embodiment, an additional anticancer agent for use in the combination therapies of the invention is a biologic agent. Biologic agents (also called biologics) are the products of a biological system, e.g., an organism, cell, or recombinant system. Examples of such biologic agents include nucleic acid molecules (e.g., antisense nucleic acid molecules), interferons, interleukins, colony-stimulating factors, antibodies, e.g., monoclonal antibodies, anti-angiogenesis agents, and cytokines. Exemplary biologic agents are discussed in more detail below and generally belong to various classes including, for example: (1) Hormones, hormonal analogues, and hormonal complexes, e.g., estrogens and estrogen analogs, progesterone, progesterone analogs and progestins, androgens, adrenocorticosteroids, antiestrogens, antiandrogens, antitestosterones, adrenal steroid inhibitors, and anti-leuteinizing hormones; and (2) Enzymes, proteins, peptides, polyclonal and/or monoclonal antibodies, such as interleukins, interferons, colony stimulating factor, etc.

In one embodiment, the biologic is an interferon. Interferons (IFN) are a type biologic agent that naturally occurs in the body. Interferons are also produced in the laboratory and given to cancer patients in biological therapy. They have been shown to improve the way a cancer patient's immune system acts against cancer cells. Interferons may work directly on cancer cells to slow their growth, or they may cause cancer cells to change into cells with more normal behavior. Some interferons may also stimulate natural killer cells (NK) cells, T cells, and macrophages which are types of white blood cells in the bloodstream that help to fight cancer cells.

In one embodiment, the biologic is an interleukin. Interleukins (IL) stimulate the growth and activity of many immune cells. They are proteins (cytokines and chemokines) that occur naturally in the body, but can also be made in the laboratory. Some interleukins stimulate the growth and activity of immune cells, such as lymphocytes, which work to destroy cancer cells.

In another embodiment, the biologic is a colony-stimulating factor. Colony-stimulating factors (CSFs) are proteins given to patients to encourage stem cells within the bone marrow to produce more blood cells. The body constantly needs new white blood cells, red blood cells, and platelets, especially when cancer is present. CSFs are given, along with chemotherapy, to help boost the immune system. When cancer patients receive chemotherapy, the bone marrow's ability to produce new blood cells is suppressed, making patients more prone to developing infections. Parts of the immune system cannot function without blood cells, thus colony-stimulating factors encourage the bone marrow stem cells to produce white blood cells, platelets, and red blood cells.

With proper cell production, other cancer treatments can continue enabling patients to safely receive higher doses of chemotherapy.

In another embodiment, the biologic is an antibody. Antibodies, e.g., monoclonal antibodies, are agents, produced in the laboratory, that bind to cancer cells. Monoclonal antibody agents do not destroy healthy cells. Monoclonal antibodies achieve their therapeutic effect through various mechanisms. They can have direct effects in producing apoptosis or programmed cell death. They can block growth factor receptors, effectively arresting proliferation of tumor cells. In cells that express monoclonal antibodies, they can bring about anti-idiotype antibody formation.

Examples of antibodies which may be used in the combination treatment of the invention include anti-CD20 antibodies, such as, but not limited to, cetuximab, Tositumomab, rituximab, and Ibritumomab. Anti-HER2 antibodies may also be used in combination with CoQ10 for the treatment of cancer. In one embodiment, the anti-HER2 antibody is Trastuzumab (Herceptin). Other examples of antibodies which may be used in combination with CoQ10 for the treatment of cancer include anti-CD52 antibodies (e.g., Alemtuzumab), anti-CD-22 antibodies (e.g., Epratuzumab), and anti-CD33 antibodies (e.g., Gemtuzumab ozogamicin). Anti-VEGF antibodies may also be used in combination with CoQ10 (e.g., for the treatment of glioma, such as glioblastoma). In one embodiment, the anti-VEGF antibody is bevacizumab. In other embodiments, the biologic agent is an antibody which is an anti-EGFR antibody e.g., cetuximab. Another example is the anti-glycoprotein 17-1A antibody edrecolomab. Numerous other anti-tumor antibodies are known in the art and would be understood by the skilled artisan to be encompassed by the present invention.

In another embodiment, the biologic is a cytokine. Cytokine therapy uses proteins (cytokines) to help a subject's immune system recognize and destroy those cells that are cancerous. Cytokines are produced naturally in the body by the immune system, but can also be produced in the laboratory. This therapy is used with advanced melanoma and with adjuvant therapy (therapy given after or in addition to the primary cancer treatment). Cytokine therapy reaches all parts of the body to kill cancer cells and prevent tumors from growing.

In another embodiment, the biologic is a fusion protein. For example, recombinant human Apo2L/TRAIL (GENETECH) may be used in a combination therapy. Apo2/TRAIL is the first dual pro-apoptotic receptor agonist designed to activate both pro-apoptotic receptors DR4 and DR5, which are involved in the regulation of apoptosis (programmed cell death).

In one embodiment, the biologic is a therapeutic nucleic acid molecule. Nucleic acid therapeutics are well known in the art. Nucleic acid therapeutics include both single stranded and double stranded (i.e., nucleic acid therapeutics having a complementary region of at least 15 nucleotides in length) nucleic acids that are complementary to a target sequence in a cell. Therapeutic nucleic acids can be directed against essentially any target nucleic acid sequence in a cell. In certain embodiments, the nucleic acid therapeutic is targeted against a nucleic acid sequence encoding a stimulator of angiogenesis, e.g., VEGF, FGF, or of tumor growth, e.g., EGFR.

Antisense nucleic acid therapeutic agents are single stranded nucleic acid therapeutics, typically about 16 to 30 nucleotides in length, and are complementary to a target nucleic acid sequence in the target cell, either in culture or in an organism.

In another aspect, the agent is a single-stranded antisense RNA molecule. An antisense RNA molecule is complementary to a sequence within the target mRNA. Antisense RNA can inhibit translation in a stoichiometric manner by base pairing to the mRNA and physically obstructing the translation machinery, see Dias, N. et al., (2002) Mol Cancer Ther 1:347-355. The antisense RNA molecule may have about 15-30 nucleotides that are complementary to the target mRNA. Patents directed to antisense nucleic acids, chemical modifications, and therapeutic uses are provided, for example, in U.S. Pat. No. 5,898,031 related to chemically modified RNA-containing therapeutic compounds, and U.S. Pat. No. 6,107,094 related methods of using these compounds as therapeutic agent. U.S. Pat. No. 7,432,250 related to methods of treating patients by administering single-stranded chemically modified RNA-like compounds; and U.S. Pat. No. 7,432,249 related to pharmaceutical compositions containing single-stranded chemically modified RNA-like compounds. U.S. Pat. No. 7,629,321 is related to methods of cleaving target mRNA using a single-stranded oligonucleotide having a plurality RNA nucleosides and at least one chemical modification. The entire contents of each of the patents listed in this paragraph are incorporated herein by reference.

Nucleic acid therapeutic agents for use in the methods of the invention also include double stranded nucleic acid therapeutics. An “RNAi agent,” “double stranded RNAi agent,” double-stranded RNA (dsRNA) molecule, also referred to as “dsRNA agent,” “dsRNA”, “siRNA”, “iRNA agent,” as used interchangeably herein, refers to a complex of ribonucleic acid molecules, having a duplex structure comprising two anti-parallel and substantially complementary, as defined below, nucleic acid strands. As used herein, an RNAi agent can also include dsiRNA (see, e.g., US Patent publication 20070104688, incorporated herein by reference). In general, the majority of nucleotides of each strand are ribonucleotides, but as described herein, each or both strands can also include one or more non-ribonucleotides, e.g., a deoxyribonucleotide and/or a modified nucleotide. In addition, as used in this specification, an “RNAi agent” may include ribonucleotides with chemical modifications; an RNAi agent may include substantial modifications at multiple nucleotides. Such modifications may include all types of modifications disclosed herein or known in the art. Any such modifications, as used in a siRNA type molecule, are encompassed by “RNAi agent” for the purposes of this specification and claims. The RNAi agents that are used in the methods of the invention include agents with chemical modifications as disclosed, for example, in U.S. Provisional Application No. 61/561,710, filed on Nov. 18, 2011, International Application No. PCT/US2011/051597, filed on Sep. 15, 2010, and PCT Publication WO 2009/073809, the entire contents of each of which are incorporated herein by reference.

Additional exemplary biologic agents for use in the methods of the invention include, but are not limited to, gefitinib (Iressa), anastrazole, diethylstilbesterol, estradiol, premarin, raloxifene, progesterone, norethynodrel, esthisterone, dimesthisterone, megestrol acetate, medroxyprogesterone acetate, hydroxyprogesterone caproate, norethisterone, methyltestosterone, testosterone, dexamthasone, prednisone, Cortisol, solumedrol, tamoxifen, fulvestrant, toremifene, aminoglutethimide, testolactone, droloxifene, anastrozole, bicalutamide, flutamide, nilutamide, goserelin, flutamide, leuprolide, triptorelin, aminoglutethimide, mitotane, goserelin, cetuximab, erlotinib, imatinib, Tositumomab, Alemtuzumab, Trastuzumab, Gemtuzumab, Rituximab, Ibritumomab tiuxetan, Bevacizumab, Denileukin diftitox, Daclizumab, interferon alpha, interferon beta, anti-4-1BB, anti-4-1BBL, anti-CD40, anti-CD154, anti-OX40, anti-OX40L, anti-CD28, anti-CD80, anti-CD86, anti-CD70, anti-CD27, anti-HVEM, anti-LIGHT, anti-GITR, anti-GITRL, anti-CTLA-4, soluble OX40L, soluble 4-IBBL, soluble CD154, soluble GITRL, soluble LIGHT, soluble CD70, soluble CD80, soluble CD86, soluble CTLA4-Ig, GVAX®, and combinations thereof which are readily apparent to one of skill in the art based on the appropriate standard of care for a particular tumor or cancer. The soluble forms of agents may be made as, for example fusion proteins, by operatively linking the agent with, for example, Ig-Fc region.

It should be noted that more than one additional anticancer agent (e.g., chemotherapeutic agents), e.g., 2, 3, 4, 5, or more, may be administered in combination with the CoQ10 formulations and radiation therapies described herein. For example, in one embodiment two additional anticancer agents may be administered in combination with CoQ10 and radiation therapy. In one embodiment, a chemotherapeutic small molecule agent, an anticancer biologic agent, radiation therapy, and CoQ10 may be administered. Appropriate doses and routes of administration of the anticancer agents provided herein are known in the art.

### Subjects for Treatment of Glioma

In some embodiments, a subject has undergone surgery for a glioma (e.g. glioblastoma, gliosarcoma) before first administration of the composition comprising Coenzyme Q10 and/or prior to initiation of radiation therapy. For example, in some embodiments, a subject has undergone surgery for a glioma (e.g. glioblastoma, gliosarcoma) less than one, two, three, four, five, six, seven or eight weeks before first administration of the composition comprising Coenzyme Q10 and/or initiation of radiation therapy. In a particular embodiment, a subject has undergone surgery for a glioma less than four weeks before first administration of the composition comprising Coenzyme Q10. In some embodiments, the subject has not undergone surgery for the glioma before first administration of the composition comprising Coenzyme Q10 and/or prior to initiation of radiation therapy.

The treatment regimens described herein may be administered to a subject that has not received any prior treatment for the glioma, or that has received only surgery for treatment of the glioma. For example, in some embodiments the subject has not been treated with an anticancer agent for the glioma before administration of the composition comprising Coenzyme Q10 is initiated and/or prior to initiation of radiation therapy. In some embodiments the subject has not been treated with temozolomide for the glioma before administration of the composition comprising Coenzyme Q10 is initiated and/or prior to initiation of radiation therapy. In some embodiments the subject has not been treated with bevacizumab for the glioma before administration of the composition comprising Coenzyme Q10 is initiated and/or prior to initiation of radiation therapy. In some embodiments the subject has not been treated with temozolomide or bevacizumab for the glioma before administration of the composition comprising Coenzyme Q10 is initiated and/or prior to initiation of radiation therapy. In some embodiments, the subject has not been treated with radiation therapy for the glioma before administration of the composition comprising Coenzyme Q10 is initiated. In some embodiments, the subject has not been treated with an anticancer agent (e.g. temozolomide and/or bevacizumab) for the glioma, and has not been treated with radiation therapy for the glioma before administration of the composition comprising Coenzyme Q10 is initiated.

Accordingly, in some aspects the disclosure relates to a method of treating a glioma in a subject comprising: (a) administering a composition comprising Coenzyme Q10 to the subject by continuous intravenous infusion; and (b) administering radiation therapy to the subject, thereby treating the glioma in the subject, wherein the subject has not been treated with an anticancer agent (e.g. temozolomide and/or bevacizumab) for the glioma.

In some aspects the disclosure relates to a method of treating a glioma in a subject comprising: (a) administering a composition comprising Coenzyme Q10 to the subject by continuous intravenous infusion; and (b) administering radiation therapy to the subject, thereby treating the glioma in the subject, wherein the subject has not been treated with radiation therapy for the glioma before administration of the composition comprising Coenzyme Q10 is initiated.

In some aspects the disclosure relates to a method of treating a glioma in a subject comprising: (a) administering a composition comprising Coenzyme Q10 to the subject by continuous intravenous infusion; and (b) administering radiation therapy to the subject, thereby treating the glioma in the subject, wherein the subject has not been treated with an anticancer agent (e.g. temozolomide and/or bevacizumab) for the glioma, and has not been treated with radiation therapy for the glioma before administration of the composition comprising Coenzyme Q10 is initiated.

In some embodiments, administration of a composition comprising a CoQ10 compound (e.g. CoQ10) and radiation therapy as described herein, reduces glioma (e.g. glioblastoma or gliosarcoma) tumor size, inhibits glioma tumor growth and/or prolongs the survival time of a glioma tumor-bearing subject relative to a subject that is administered the pharmaceutical composition comprising a CoQ10 compound (e.g. CoQ10) alone, or the radiation therapy alone. For example, in some embodiments, the subject exhibits an increase in one or more of overall survival and progression free survival relative to a subject that is administered the pharmaceutical composition comprising a CoQ10 compound (e.g. CoQ10) alone, or the radiation therapy alone. Overall survival (OS) is determined by measuring from the start date of Coenzyme Q10 treatment to the date of death or date of last follow-up (for subjects who have not died or are lost to follow up). In some embodiments, the median overall survival time for a population of subjects is at least 1, 2, 3, 4, 5, 6, 7, 8, 9, 10, 1112, 13, 14, 15, 16, 17, 18, 19, 20, 21, 22, 23, 24, 25, 26, 27, 28, 29, 30, 31, 32, 33, 34, 35 or 36 months. Progression free survival (PFS) is determined by measuring from the start date of Coenzyme Q10 treatment to the date of progression of the glioma. In some embodiments, the median progression free survival time for a population of subjects is at least 1, 2, 3, 4, 5, 6, 7, 8, 9, 10, 1112, 13, 14, 15, 16, 17, 18, 19, 20, 21, 22, 23, 24, 25, 26, 27, 28, 29, 30, 31, 32, 33, 34, 35 or 36 months. In some embodiments, the subject was not previously treated with radiation therapy for the glioma and/or a chemotherapeutic agent for the glioma. In some embodiments, the subject has failed at least one prior cancer therapeutic regimen as compared to an appropriate control.

Gliomas for treatment using the methods of the invention include, but are not limited to, ependymomas, astrocytomas (e.g. glioblastoma, gliosarcoma), oligodendrogliomas, brainstem gliomas, optic nerve gliomas, and mixed gliomas (e.g. oligoastrocytomas) which contain cells from different types of glia. In a particular embodiment, the glioma is a glioblastoma. In some embodiments, the glioma is a low-grade glioma. In other embodiments, the glioma is a high-grade glioma.

In some embodiments, the compositions and methods provided herein are for the treatment of glioma (e.g. glioblastoma) in a subject wherein the subject has previously failed at least one prior (e.g., 1, 2, 3, 4, 5, 6, 7, 8, 9, 10, or more) cancer therapeutic regimen, e.g., radiation therapy or a chemotherapeutic regimen for the glioma. In a particular embodiment, the chemotherapeutic regimen comprises administering TMZ to the subject. In certain embodiments, the subject has failed treatment for the glioma with at least one additional cancer therapeutic agent, e.g. bevacizumab (Avastin). A subject who has failed a cancer therapeutic regimen does not achieve at least stable disease or loses stable disease as defined by RECIST 1.1 criteria after a short period in at least one target lesion during or after the treatment with the chemotherapeutic regimen. In certain embodiments, a short period is less than 6 months. In certain embodiments, a short period is less than 5 months. In certain embodiments, a short period is less than 4 months. In certain embodiments, a short period is less than 3 months. In certain embodiments, a short period is less than 2 months. In certain embodiments, a short period is less than 1 month. In certain embodiments, a short period is less than 2 weeks. A subject who has failed a cancer therapeutic regimen includes a subject who has progressive disease during treatment or shortly after the end of treatment with a cancer therapeutic regimen. In a preferred embodiment, the subject who has failed a cancer therapeutic regimen still has, or is suspected of having, the primary tumor. It is understood that it may not be possible or desirable to specifically identify the primary tumor, particularly when a subject presents with metastatic disease. In a preferred embodiment, the subject who has failed a cancer therapeutic regimen still has a tumor at the site of the primary tumor, i.e., in the organ in which the primary tumor arose. In certain embodiments, the primary tumor is a solid tumor.

A subject who has failed a cancer therapeutic regimen has not been cured of the glioma (e.g. glioblastoma) being treated or according to a clinical definition, e.g., achieving complete remission in target or non-target lesions per the RECIST 1.1 criteria for an extended period after the conclusion of treatment of the glioma, e.g., for at least one year, at least 5 years, at least 10 years. For example, a subject treated for a glioma (e.g. glioblastoma) who is cured, i.e., who has achieved complete remission, has a greater chance of suffering from a distinct cancer later in life. A subject successfully treated for a glioma (i.e., a subject who achieves clinical remission for a sufficient time to be considered cured, e.g., at least 5 years of clinical remission) who later develops a second cancer is not considered to have failed the first cancer therapeutic regimen.

A subject who has failed a cancer therapeutic regimen may have also undergone other treatments for the glioma (e.g. glioblastoma) in conjunction with an anticancer agent including surgery for tumor resection and/or radiation therapy. The subject may have undergone bone marrow transplant or other procedures. It is understood that failure of a cancer therapeutic regimen may be due, at least in part, to a failure of one or more interventions other than the anticancer agent.

Failure of a cancer therapeutic regimen can result from the subject suffering from a dose limiting toxicity, e.g., a grade IV toxicity or a lower grade toxicity that cannot be tolerated by the subject or resolved with other interventions, e.g., anti-nausea agents, stimulators of red blood cell production, agents to normalize clotting, agents to reduce immune/allergic response, etc., depending on the specific toxicity. It is understood that such dose limiting toxicities can result in a shortened or incomplete cancer therapeutic regimen being administered to the subject, resulting in reduced efficacy of the agent.

Reference will now be made in detail to preferred embodiments of the invention. While the invention will be described in conjunction with the preferred embodiments, it will be understood that it is not intended to limit the invention to those preferred embodiments. To the contrary, it is intended to cover alternatives, modifications, and equivalents as may be included within the spirit and scope of the invention as defined by the appended claims.

Each patent, publication, and reference cited herein is incorporated herein by reference in their entirety. Further, WO 2008/116135 (PCT Appln. No. PCT/US2008/116135), WO2009/073843 (PCT Appln. No. PCT/US2008/085669), WO2010/132507 (PCT Appln. No. PCT/US2010/034453), WO2011/11290 (PCT Appln. No. PCT/US2011/028042), WO2012/174559 (PCT Appln. No. PCT/US2012/043001), US Patent Application Publication No.: US2011/0027247, US Patent Application Publication No.: US2015/0157559, are hereby incorporated by reference in their entirety.

### EXAMPLES

### Example 1: Coenzyme Q10 Significantly Prolongs Survival in a C6 Rat Glioma Model

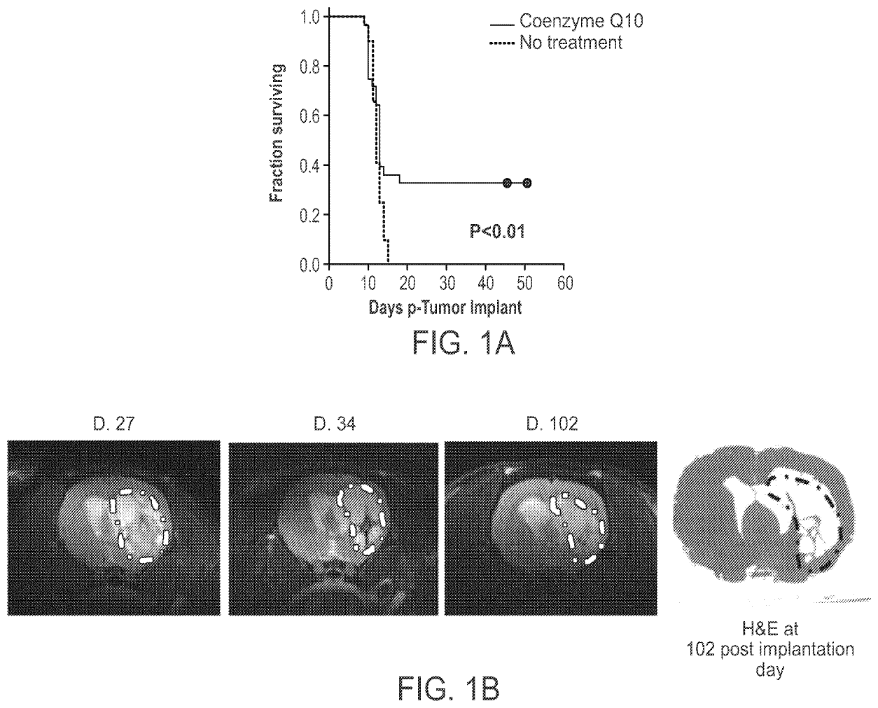

10⁶ C6 glioma cells were implanted into the right striatum of Wistar rats. Four days after implantation, animals started to receive saline (n=32) or 50 mg/kg of Coenzyme Q10 (n=21), twice a day, i.p. for 45 days. Surviving animals remained in the study until Day 102. As shown in FIG. 1A, over 25% of rats treated with Coenzyme Q10 survived 45 days, considered long-term survival in this model (p<0.01, log rank test). The drug effect persisted after treatment was withdrawn, demonstrated by the continued shrinkage of the tumor over time as shown in FIG. 1B (marked as dashed line).

### Example 2: Assessment of Superoxide Production in Glioma Cells and Non-Cancerous Cells Treated with Coenzyme Q10

We assessed the effects of Coenzyme Q10 on mitochondrial superoxide production in in vitro cocultures of glioma and non-cancerous cells, i.e. rodent C6 glioma, rodent NIH3T3 non-cancerous fibroblast cells, human U251 glioma, and primary human non-cancerous astrocytes.

For the rodent cells, NIH3T3 and C6 cells were initially seeded in a 1:1 ratio. After 72 h or 144 h incubation with Coenzyme Q10 (0 μM, 72 μM, 115 μM, 230 μM, 345 μM or 460 μM), the number of C6 or NIH3T3 cells in co-culture was generated by flow-cytometry. Images were taken at 72 hrs post-dosing (0 μM, 230 μM or 460 μM) using a Leica CTR5000. NIH3T3 and C6 cells co-cultured with certain conc. of Coenzyme Q10 for 144 hours were subjected to imaging and flow-cytometry. Superoxide level was detected by Mitosox staining. The percentage of cells harboring positive or negative levels of superoxide from C6 (GFP+) or NIH3T3 (GFP−) populations were determined by flow cytometry.

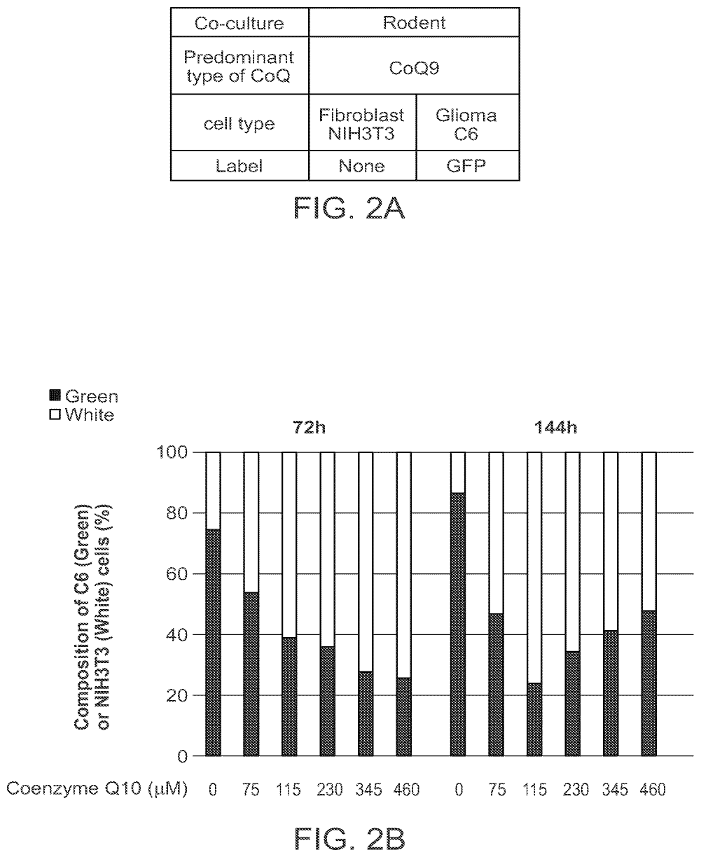

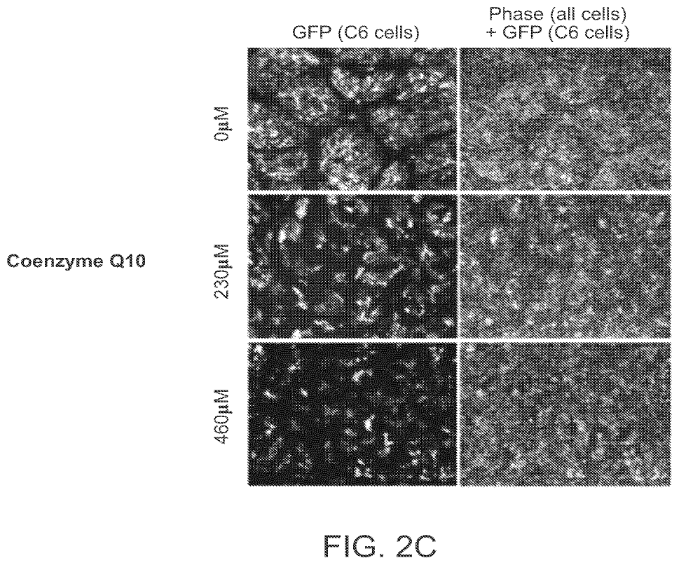

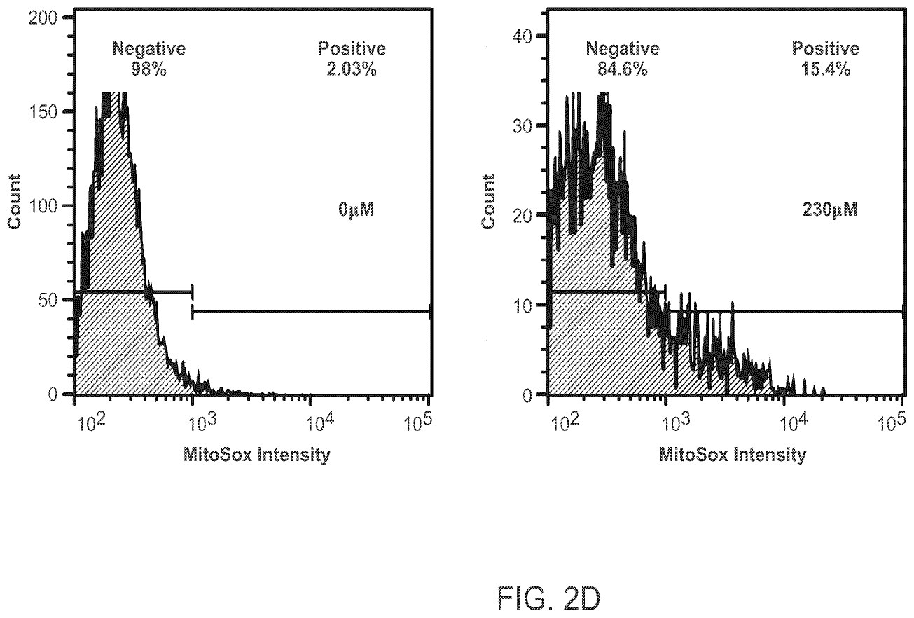

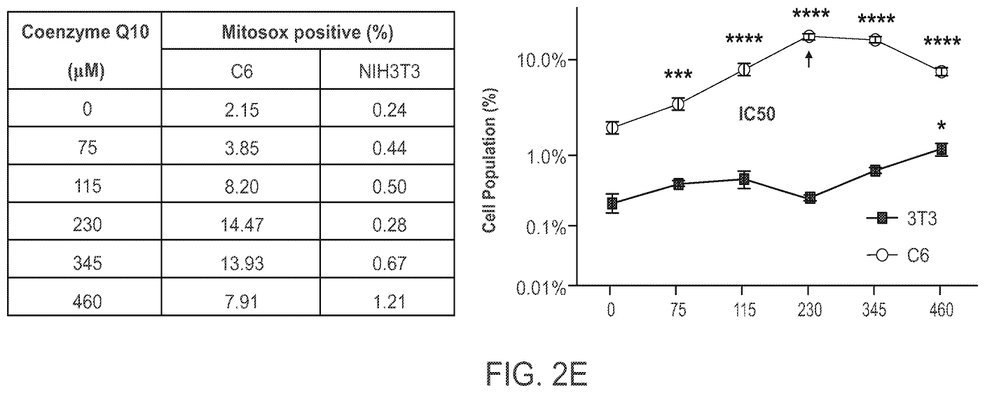

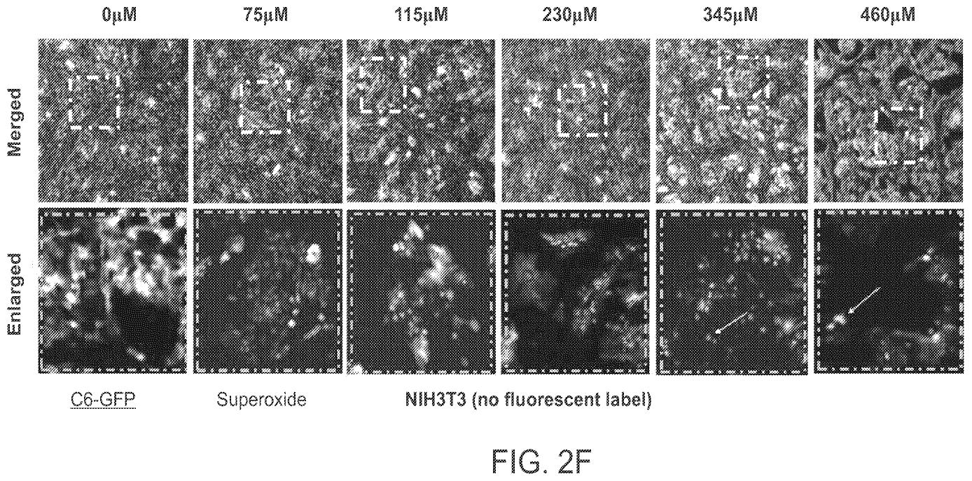

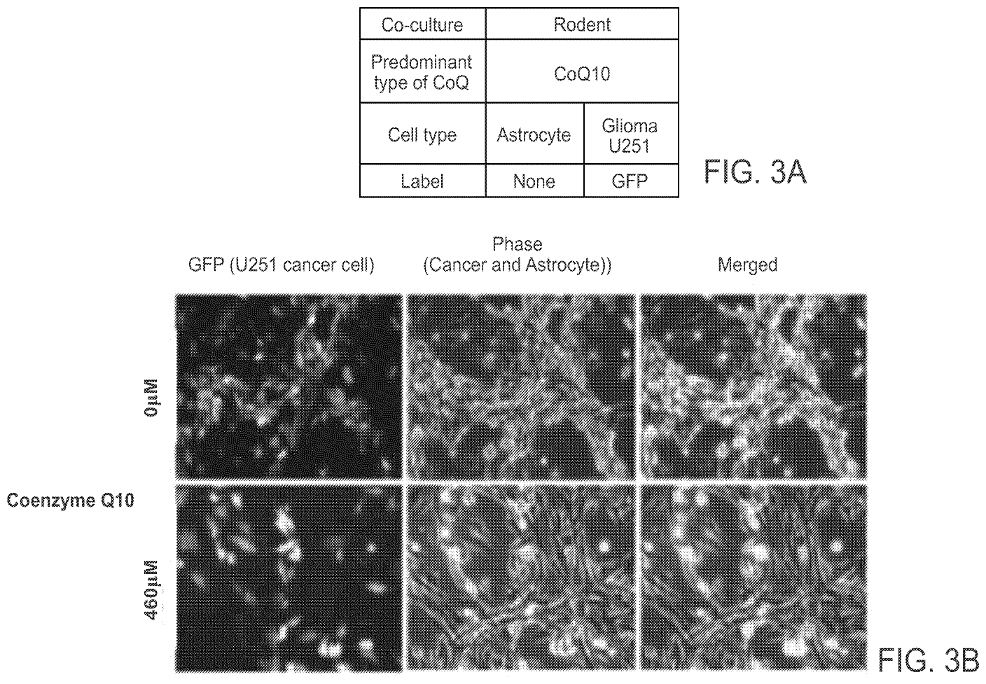

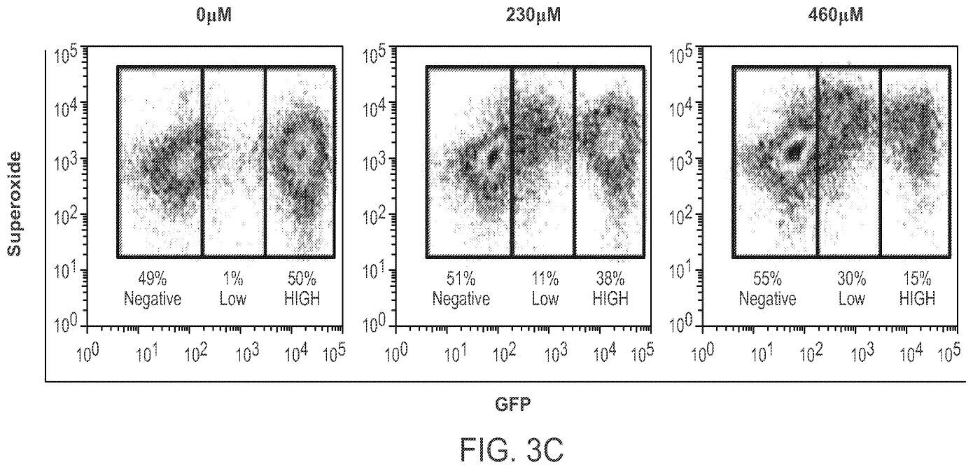

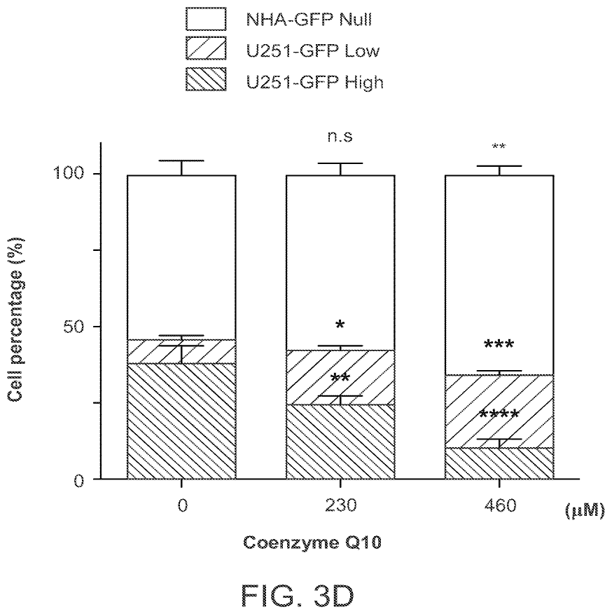

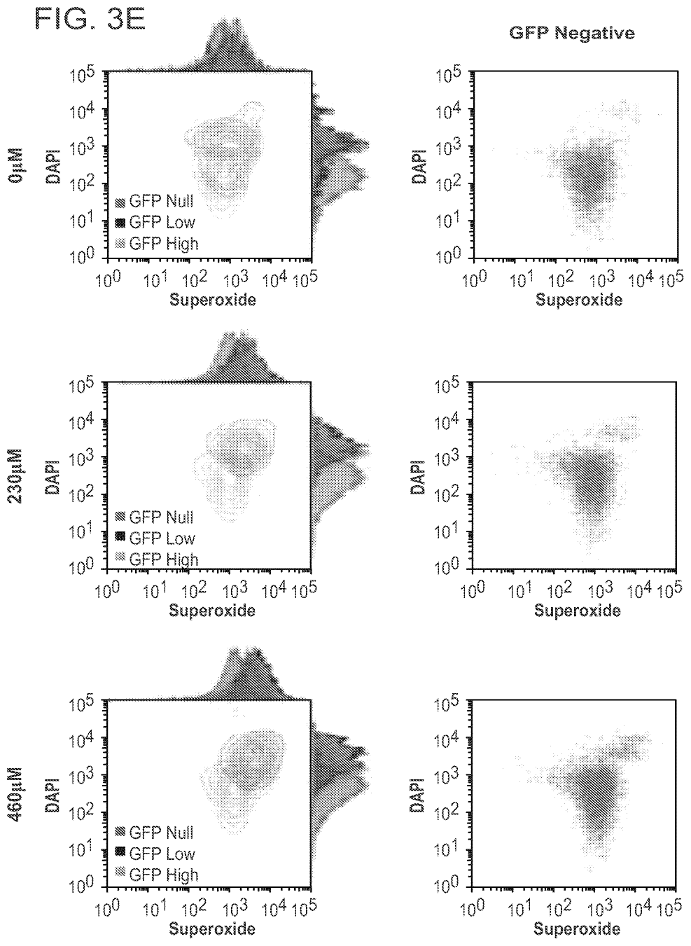

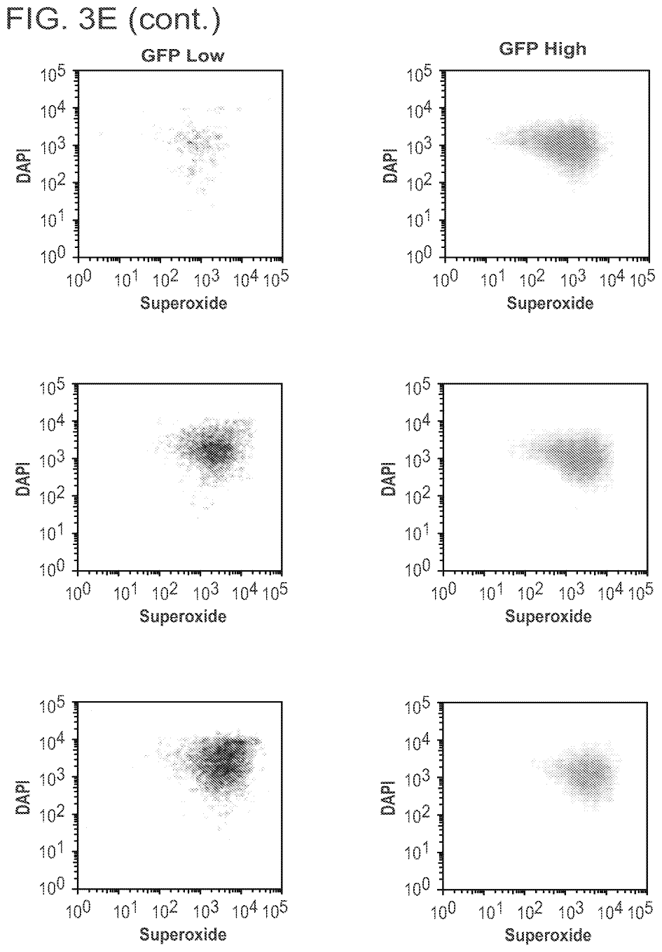

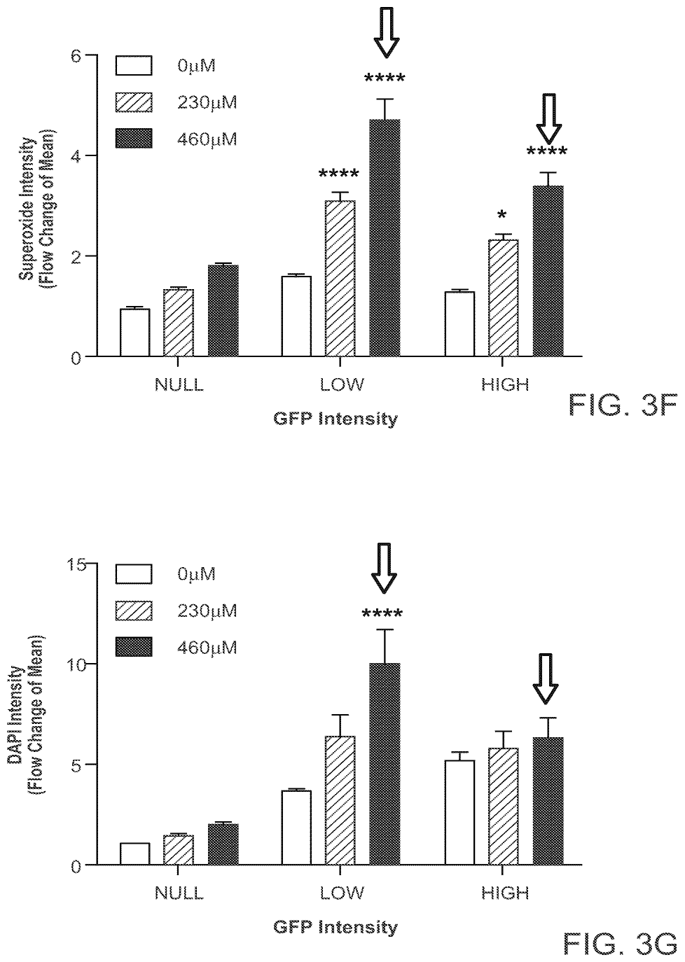

In the human cell model, NHA (non-label) and U251 (GFP-labelled) cells were co-cultured at a designated cell density. Phase and fluorescent images of U251/NHA Co-cultures of U251/NHA cells were treated with 0 or 460 μM Coenzyme Q10 and detected by phase and fluorescent imaging. Coenzyme Q10 treatment decreased glioma cell levels with a relative sparing of NHA cells. See FIG. 2 B. Cells were also analyzed by flow cytometric analysis (scatter plot) of GFP and superoxide for U251/NHA cocultures. Cell populations were characterized based by GFP intensity (GFP-negative, GFP-low, and GFP-high). An increased percentage of GFP-low relative to GFP-high cells was observed with increasing dose. See FIG. 3C. Quantification of cell percentages illustrated the increased number of GFP-low cells with increased dose. See FIG. 3D. Flow cytometric analysis of superoxide and DAPI for NHA-U251/NHA co-cultures treated with 0, 230, or 460 μM Coenzyme Q10 indicated an increase in superoxide values for both GFP-low and GFP-high cells, while insignificant changes were noted for GFP-negative (non-cancerous NHA) cells. See FIGS. 3E and 3F. In contrast, DAPI levels were only significantly elevated in GFP-low cells, consistent with this population representing dying cells (as shown in FIG. 3G).

Interestingly, the peak effect of differences in superoxide levels and cell growth occurred at the same dosage of Coenzyme Q10 (230 μM). A similar differential (glioma selective) sensitivity to Coenzyme Q10 was observed in the human derived model. Basal superoxide levels for U251 human glioma cells were 1.5-fold higher compared to the human astrocytes, and this difference increased to over 4-fold upon treatment with Coenzyme Q10, suggesting Coenzyme Q10 exploits differential redox vulnerabilities between U251 and astrocytes to mediate its anti-cancer activity.

### Example 3: a Phase 1 Study of Coenzyme Q10 Plus Vitamin K in Subjects with High-Grade Glioma that has Recurred on a Bevacizumab-Containing Regimen

High grade gliomas (HGG) are characterized by dysregulated metabolism, utilizing glycolysis to support unrestricted growth. The patients in this study were administered a CoQ10-lipid conjugate in which oxidized CoQ10 was incorporated into a mixture of mitochondria compatible lipids, enabling delivery of supraphysiological concentrations of CoQ10 to the mitochondria. In vitro, Coenzyme Q10 produces a differential inhibitor effect on neoplastic C6 and U251 glioma cells versus normal human astrocytes, as shown in Example 2 above. C6 and U251 cells exposed to Coenzyme Q10 have significantly more induction of mitochondrial superoxide than non-neoplastic cells, as also shown in Example 2. As shown in Example 1, in vivo, Coenzyme Q10 significantly prolongs survival in the C6 rat glioma model, with 25% of animals with confirmed tumor living long-term. These data suggest that Coenzyme Q10 may have activity in high-grade gliomas.

This study was a phase I trial of Coenzyme Q10 to assess for safety and tolerability of Coenzyme Q10 with vitamin K in patients with HGGs that were progressive after a bevacizumab (BEV) containing regimen. Elevated INR without clinical bleeding was identified as a side effect in prior studies. Therefore, this study used 10 mg vitamin K prophylactically.

Design: Modified toxicity probability interval design (mTPI) enrolling patients sequentially and monitoring through a 28-day DLT period. 1 dose escalation and 2 dose de-escalations allowed.

Key Inclusion Criteria: HGG (GB, Gliosarcoma, AA, AO) sp radiation with TMZ in recurrence after treatment with BEV. Evidence of leptomeningeal spread was allowed. >18 w KPS>60. Normal coagulation labs.

Key Exclusion Criteria: History of spontaneous or tumor related intracerebral hemorrhage. Uncontrolled coagulopathies or use of anti-coagulants.

The patient population is described in Table 2 below.

TABLE 2
Demographics of patient population.

| Characteristics | N |
|---|---|
| Eligible & started treatment | 12 |
| Eligible & evaluable_{(completed 28-day DLT period or had DLT)} | 9 |
| Progression while on protocol | 2 |
| Discontinued for G2 asymptomatic ICH | 1 |
| Gender | |
| Female | 3 |
| Male | 6 |
| Median age | 55 yo (27-67) |
| Median KPS | 70 (60-90) |
| Histology | |
| Primary Glioblastoma | 8 |
| GBM after initial AA diagnosis | 1 |
| MGMT Status | |
| Methylated | 5 |
| Unmethylated | 4 |
| Unknown | 0 |
| IDH mutation | |
| Not present | 6 |
| Present | 1 |
| Unknown | 2 |

Coenzyme Q10+vitamin K was safe and feasible in patients with HGG recurrent after a BEV-containing regimen. 4 patients were treated at the top dose of 171 mg/kg. One patient experienced DLT of grade 3 AST and ALT elevation, which occurred at the 171 mg/kg dose. The most common grade 1-2 AEs possibly, probably, or definitely related to drug were elevated AST, rash, and fatigue, each occurring in 3 patients.

The progression free survival (PFS) for eligible and evaluable patients was 34 days (CI of mean 8.9). The overall survival (OS) for eligible and evaluable patients was 128 days (95% CI: 48-209). Two patients survived more than 1 year, and one patient was still alive at 8 months.

G1 elevated JNR was seen in only one patient in this study. The median KPS on this study was relatively low compared to other studies in this space. Two patients on this study had evidence of CSF disease spread at the time of study enrollment. mOS for patients in this study was comparable to other post-BEV studies (Nowosielski M, et al., 2014, Neurology 82:1684-1692; Reardon D A et al., 2011, Cancer 117 (53):5351-5358.).

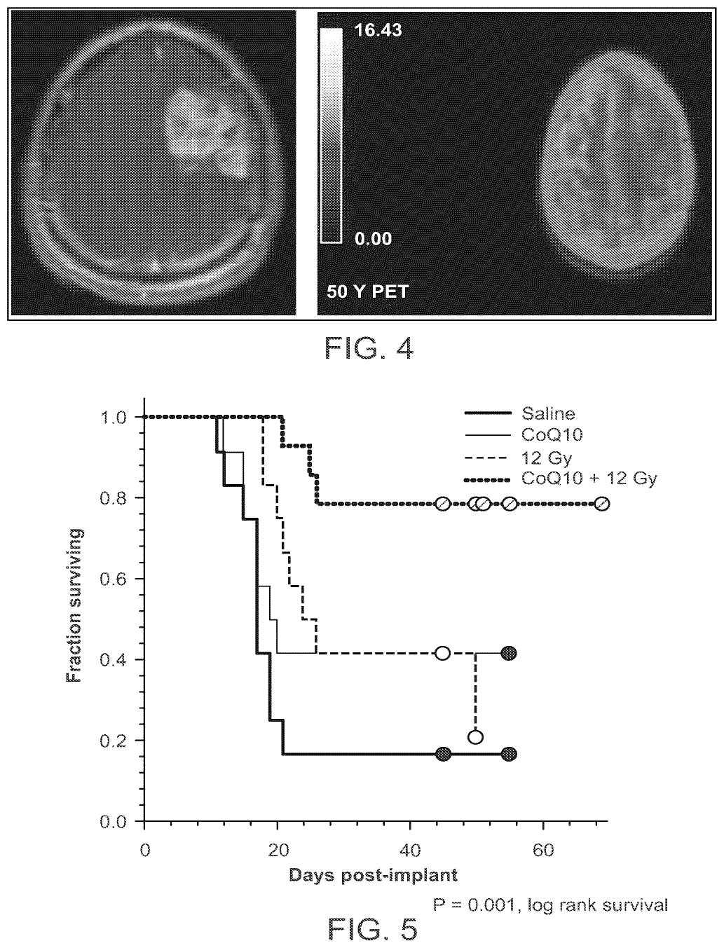

One patient received FDG-PET after 6 weeks in the study. See FIG. 4. The PET demonstrated decreased glucose uptake in the area of his prior surgery, but MRI at 8 weeks demonstrates a large recurrent tumor. This suggests that Coenzyme Q10 may be altering the metabolism of tumor. While most patients had worsening imaging at 1-month, standard MRI may not allow us to adequately monitor patients being treated with metabolic therapies.

In conclusion, we established that continuous infusion of Coenzyme Q10+vitamin K is safe and well-tolerated in doses up to 171 mg/kg. Although we did not see RANO responses in this treatment-refractory group, one patient's PET-CT suggests Coenzyme Q10 may be altering tumor metabolism.

### Example 4: a Coenzyme Q10 (CoQ10)-Containing Lipid Nanodispersion, Increases Radiation Effects and Prolongs Survival in a Rodent Glioblastoma Model

Methodology: 1×10⁶ luciferase labeled C6 cells were implanted into the right striatum of Sprague Dawley rats. 4 days post-implantation, 50 rats were randomized into one of four groups:
(i) Saline injection ip bid; (12 rats)
(ii) Coenzyme Q10 50 mg/kg ip bid to continue up to 35 days; (12 rats)
(iii) 12 Gy radiotherapy (RT) to be administered on Day 8 post-implant with saline injection (12 rats); and
(iv) Coenzyme Q10+RT (14 rats).

Tumor-bearing rats were monitored until death or Day 50.

Results: Log rank survival analysis indicated a marked enhancement of median survival with the addition of Coenzyme Q10 to RT. While neither RT nor Coenzyme Q10 enhanced median survival relative to saline, the combination was markedly more effective (median survival of 17, 19, 24 and >50 days for saline, Coenzyme Q10, RT and combination, respectively, p<0.001). This was also reflected in increase in frequency of long term survival (LTS), which was over 70% (11 of 14 rats) in the combination group (p<0.01 compared to control). See FIG. 5. The combination of CoQ10 and RT also exhibited the highest rates of long term survival, as shown in Table 3 below. Long term survivors in this model are considered to be subjects that have survived for 45 days.

TABLE 3
Long Term Survivors

| Treatment Group | Long Term Survivors (%) |
|---|---|
| Saline | 2/12 (17%) |
| Radiotherapy | 5/12 (42%) |
| CoQ10 | 5/12 (42%) |
| CoQ10 + Radiotherapy | 11/14 (79%) |

Conclusion: Coenzyme Q10 significantly enhanced the therapeutic efficacy of radiation in this rat model of glioblastoma. Indeed, there was both an effect on median survival as well as an enhancement of long term survival (LTS) with combination treatment. The low toxicity profile of Coenzyme Q10 and its potential protective effects on normal cells may offer a unique strategy with which to enhance radiation therapy.

### Example 5: a Human Clinical Trial of Combination Therapy of Coenzyme Q10 and Radiation in Patients Diagnosed with Glioma

The primary objective of this study is to assess the efficacy of Coenzyme Q10 and Vitamin K1 administered neo-adjuvantly and concurrently with standard radiation therapy (RT) and temozolomide (TMZ) in subjects with newly diagnosed glioblastoma (GB) and gliosarcoma as measured by overall survival. The secondary objectives of this study are to assess the effect, safety, and tolerability of Coenzyme Q10 and Vitamin K1 administered neo-adjuvantly and concurrently with standard RT and TMZ on progression free survival in subjects with newly diagnosed GB and gliosarcoma.

This is a single arm, non-randomized, open label Phase 2 therapeutic study that will assess the effects of adding Coenzyme Q10 onto a conventional treatment framework of RT and concurrent TMZ chemotherapy for subjects with newly diagnosed GB and gliosarcoma.

On Day 1, subjects will receive their first 96 hour (h) continuous intravenous infusion of Coenzyme Q10 at a dose of 110 mg/kg/week (wk). Coenzyme Q10 nanosuspension injection (40 mg/mL) will be administered starting within 4 wk of surgery. Subjects will receive a Coenzyme Q10 110 mg/kg/wk continuous intravenous infusion once weekly for 8 wk (i.e., through Day 54). To monitor and mitigate Coenzyme Q10-associated coagulopathies, the prothrombin time, partial thromboplastin time, International Normalized Ratio, and platelet count must be assessed prior to each dose of Coenzyme Q10. Prophylactic Vitamin K1 will be given to all subjects prior to the beginning of each week of therapy.

After 2 wk of treatment with Coenzyme Q10 (i.e. on Day 15), subjects will start concurrent standard RT and TMZ 75 mg/m² once per day×42 d (i.e., until Day 54). Subjects will have a Safety Follow-up (FU) visit 28 d (±5 d) after the last RT/TMZ treatment.

The investigational drug product to be used in this study is a 4% (w/v) sterile Coenzyme Q10 aqueous nanosuspension. It is intended to deliver a high dose of the active drug, Coenzyme Q10 (Ubidecarenone: 4000 mg [100 mL]), undiluted when administered as a single, slow 96-h IV infusion. The drug product is produced using a microfluidization process, which results in a stable nanosuspension with a mean particle size of 30 to 80 nm. The nanosuspension formulation comprises 40 mg/mL (36.0 to 44.0 mg/mL) Coenzyme Q10.

The dose of Coenzyme Q10 used for this study will be 110 mg/kg/wk. The appropriate amount of Coenzyme Q10 will be administered at the dose levels specified in Table 4 below. The volume of each dose will be based on the subject's weight in kilograms. In the event of an SAE or AE that is Grades 1 or 2 or an AE that is Grades 1 through 4 that can be directly attributable to Coenzyme Q10, there will be 1 dose reduction allowed from 110 to 88 mg/kg/wk. The AE must be resolved to Grade 1 before the study drug can be resumed.

TABLE 4
Coenzyme Q10 Nanosuspension Injection Doses

| | Dose (Monday or Tuesday) | | |
|---|---|---|---|
| | Loading dose to be | | Total Coenzyme |
| Dose | infused over 1 h | Remainder to be | Q10 dose |
| Level | (8.2% of dose) | infused over 95 h. | per week |
| −1^{a} | 7.22 mg/kg | 80.78 mg/kg | 88 mg/kg |
| 1^{b} | 9.02 mg/kg | 100.98 mg/kg | 110 mg/kg |

^{a}Dose will be used when dose reduction is required.
^{b}Starting dose
Abbreviations: h = hour(s)

Blood samples will be collected at each visit during the treatment period (i.e., Days 1, 8, 15, 22, 29, 36, 43, 50, and 54) for PK and pharmacodynamics assessments. All subjects who come off study will be followed for overall survival and subsequent anti-cancer therapies every 12 wk (±2 wk) for 5 years (y) unless withdrawal, study completion/termination, or death.

The primary endpoint is overall survival as determined by measuring from start date of Coenzyme Q10 to the date of death or date of last follow-up (for subjects who have not died or are lost to follow up). The treatment will be considered promising if median overall survival time is at least 22 months, and the treatment will be considered to not be promising if the median survival time is <14 months. To assess whether the treatment regimen is promising, the median overall survival time will be calculated. Overall survival time for each subject will be calculated as the number of days between the date of first dose of Coenzyme Q10 and the date of death or latest follow-up converted to months by dividing by 30 d.

The secondary endpoints are progression free survival at 6 months, defined as the proportion of subjects who have met response assessment in neuro-oncology (RANO) criteria for complete response, partial response, or stable disease at 6 months following initiation of Coenzyme Q10; and toxicity and tolerability.

The RANO response criteria are as follows:
Complete response (CR) requires all of the following: complete disappearance of all enhancing measurable and non-measurable disease; no new lesions; stable or improved non-enhancing (T2/FLAIR) lesions; and subject must be off corticosteroids or on physiologic replacement doses only, and stable or improved clinically.
Partial response (PR) requires all of the following: ≥50% decrease, compared with baseline, in the sum of products of perpendicular diameters of all measurable enhancing lesions; no progression of non-measurable disease; no new lesions; stable or improved non-enhancing (T2/FLAIR) lesions on same or lower dose of corticosteroids compared with baseline scan; and subject must be on a corticosteroid dose not greater than the dose at time of baseline scan and is stable or improved clinically.
Progression (PD) is defined by any of the following: ≥25% increase in sum of the products of perpendicular diameters of enhancing lesions (compared with baseline if no decrease) on stable or increasing doses of corticosteroids; a significant increase in T2/FLAIR non-enhancing lesions on stable or increasing doses of corticosteroids compared with baseline scan or best response after initiation of therapy, not due to comorbid events; the appearance of any new lesions; clear progression of non-measurable lesions; or definite clinical deterioration not attributable to other causes apart from the tumor, or to decrease in corticosteroid dose. Failure to return for evaluation as a result of death or deteriorating condition should also be considered as progression.
Stable Disease: Does not qualify as CR, PR, or PD.
Unknown: Progression has not been documented and one or more measurable and evaluable sites have not been assessed.

An MRI (±PET) will be done 30±3 d after the completion of chemo-radiation. Follow up MRIs will be done per standard of care (approximately every 8 to 12 wk), with an MRI (±PET) occurring 6 mo post the start of RT, until progression or withdrawal of consent.

The inclusion and exclusion criteria for this study are provided below.

### Inclusion Criteria

Subjects with newly diagnosed pathologically verified GB (by the World Health Organization 2016 criteria or using molecular features defined in C-IMPACT) or gliosarcoma with any evidence of residual disease (enhancing or fast fluid-attenuated inversion-recovery [FLAIR]).
No prior RT, chemotherapy, immunotherapy or targeted agents administered specifically for the lesion being treated.

$$Age \geq 18 y .$$

Life expectancy≥3 mo and in the opinion of the investigator is expected to complete the concurrent phase of chemoradiation without the use of bevacizumab or its biosimilars.

$$Karnofsky performance score \geq 60.$$

Adequate organ and marrow function as follows (all required):

$$Absolute neutrophil count \geq 1.5 k / uL$$

$$Platelets \geq 10 \; 0 k / uL$$

$$Hemoglobin \geq 9 g / dL$$

Serum creatinine≤1.8 mg/dL or creatinine clearance>50 mL/min

$$Bilirubin \leq 1.5 mg / dL$$

$$Alanine aminotransferase ( ALT ) \leq 3. \times upper limit of normal ( ULN )$$

$$Aspartate transaminase ( AST ) \leq 3. \times ULN$$

$$PT \leq 1.5 \times ULN$$

$$INR \leq 1.5 \times ULN ; PTT \leq 1.5 \times ULN$$

Ability for subject to understand and the willingness to sign a written ICF.
Subjects of childbearing potential must agree to use hormonal or barrier birth control with spermicidal gel to avoid pregnancy during the study.
Be at least 14 d out from surgery.

### Exclusion Criteria

No evidence of residual tumor (enhancing or FLAIR)
History of clinically significant tumor-related cerebral hemorrhage.
Any of the following cardiac history:
   Active heart disease including myocardial infarction within previous 3 months
   Symptomatic coronary artery disease
   Clinically significant arrhythmias not controlled by medication
   Unstable angina pectoris
   Uncontrolled or symptomatic congestive heart failure (New York Heart Association Class III and IV)
Uncontrolled or severe coagulopathies or a history of clinically significant bleeding within the past 6 mo, including any of the following, but not limited to:
Severe epistaxis
Hemoptysis
Hematochezia
Hematuria
GI bleeding
Spontaneous or tumor-related intracranial hemorrhage
Known predisposition for bleeding such as von Willebrand's disease or other such condition(s).
Uncontrolled concurrent illness that would limit compliance with study requirements, including any of the following, but limited to:
Uncontrolled infection
Psychiatric illness/social situations
Prior malignancy except for non-melanoma skin cancer and carcinoma in situ (of the cervix or bladder), unless diagnosed and definitively treated more than 3 y prior to first dose of study drug.
Receiving any of the following medications:
   Therapeutic doses of any anticoagulant, including low-molecular weight heparin. Concomitant use of warfarin, even at prophylactic doses, is prohibited.
   Digoxin, digitoxin, lanatoside C, or any type of digitalis alkaloids.
   Antiangiogenic drugs (i.e., Avastin) either in the past 2 wk or if anticipated within the next 2 wk of informed consent.
Known allergy to CoQ10.
Known allergy or adverse reaction to oral, subcutaneous, or intravenous Vitamin K1.
Pregnant or lactating.
Known to be positive for the human immunodeficiency virus (HIV). Note: HIV testing is not required for eligibility, but if performed previously and was positive, the subject is ineligible.

1. A method of treating a glioma in a subject comprising:
(a) administering a composition comprising Coenzyme Q10 to the subject by continuous intravenous infusion; and
(b) administering radiation therapy to the subject,
wherein the composition comprising Coenzyme Q10 is administered to the subject by continuous intravenous infusion for at least 24 hours before the radiation therapy is initiated, thereby treating the glioma in the subject.

2. The method of claim 1, wherein the composition comprising Coenzyme Q10 is administered to the subject by continuous intravenous infusion for at least 48 hours, at least 72 hours, or at least 96 hours before the radiation therapy is initiated.

3. The method of claim 1, wherein at least two doses of the composition comprising Coenzyme Q10 are administered to the subject by continuous intravenous infusion before the radiation therapy is initiated.

4. The method of claim 3, wherein each of the at least two doses is administered once per week.

5. The method of claim 1, wherein the radiation therapy is initiated at least one week after administration of the composition comprising Coenzyme Q10 is initiated.

6. (canceled)

7. The method of claim 1, wherein the glioma is an anaplastic astrocytoma or a glioblastoma.

8. The method of claim 7, wherein the glioblastoma is a gliosarcoma.

9. The method of claim 1, wherein the subject has undergone surgery for the glioma before administration of the composition comprising Coenzyme Q10 is initiated.

10. The method of claim 1, wherein the subject has not been treated with an anticancer agent for the glioma before administration of the composition comprising Coenzyme Q10 is initiated.

11. The method of claim 10, wherein the anticancer agent is selected from temozolomide (TMZ) and bevacizumab.

12. The method of claim 1, wherein the subject has not been treated with radiation therapy for the glioma before administration of the composition comprising Coenzyme Q10 is initiated.

13. The method of claim 1, wherein the subject demonstrates a clinical benefit selected from the group consisting of stable disease per RECIST 1.1 criteria, partial response per RECIST 1.1 criteria, and complete response per RECIST 1.1 criteria as a result of administration of the composition comprising the Coenzyme Q10 compound and the radiation therapy.

14-17. (canceled)

18. The method of claim 1, wherein the glioma comprises a Stage I tumor, a Stage II tumor, or a Stage III tumor.

19-20. (canceled)

21. The method of claim 1, wherein the glioma comprises a Stage IV tumor.

22. The method of claim 1, wherein the glioma is a low grade glioma.

23. The method of claim 1, wherein the glioma is a high grade glioma.

24-25. (canceled)

26. The method of claim 1, wherein the Coenzyme Q10 is administered at a dose of at least 50 mg/kg/week, at least 66 mg/kg/week, at last 88 mg/kg/week or at least 110 mg/kg/week.

27. (canceled)

28. The method of claim 1, wherein the Coenzyme Q10 is administered at a dose selected from the group consisting of at least 50 mg/kg/dose, at least 66 mg/kg/dose, at least 88 mg/kg/dose, or at least 110 mg/kg/dose.

29-31. (canceled)

32. The method of claim 1, wherein the radiation therapy is administered at a total dose of 0.1 to 100 Gy.

33. The method of claim 1, wherein the radiation therapy is administered at a rate of 10 to 5000 cGy/min.

34. The method of claim 1, wherein the subject is human.

35. The method of claim 1, further comprising administering an additional cancer therapy to the subject.

36. The method of claim 35, wherein the additional cancer therapy comprises exposing the glioma to an alternating electrical field.

37. The method of claim 35, wherein the additional cancer therapy comprises administration of one or more anticancer agents to the subject.

38. The method of claim 37, wherein the one or more anticancer agents is selected from the group consisting of temozolomide (TMZ) and bevacizumab.

39-40. (canceled)

41. The method of claim 1, wherein the method further comprises administering Vitamin K1 to the subject.

42. The method of claim 1, wherein the subject exhibits an increase in one or more of overall survival and progression free survival relative to a subject that is administered the pharmaceutical composition comprising Coenzyme Q10 alone, or the radiation therapy alone.

43. The method of claim 1, wherein the composition comprising Coenzyme Q10 and the radiation therapy have a synergistic effect in treating the glioma.
